(19) United States
(12) Patent Application Publication
(10) Pub. No.: US 2024/0013862 A1
(43) Pub. Date: Jan. 11, 2024

(54) METHODS TO IDENTIFY NOVEL INSECTICIDAL PROTEINS FROM COMPLEX METAGENOMIC MICROBIAL SAMPLES

(71) Applicant: Zymergen Inc., Emeryville, CA (US)

(72) Inventors: Oliver LIU, San Francisco, CA (US); Sangita GANESH, Emeryville, CA (US)

(21) Appl. No.: 18/022,848

(22) PCT Filed: Sep. 29, 2021
(86) PCT No.: PCT/US2021/052628

§ 371 Date: Feb. 23, 2023

Related U.S. Application Data
(60) Provisional application No. 63/085,553, filed on Sep. 30, 2020.

(51) Int. Cl. G16B 35/00; G16B 15/30; C12N 15/10

(52) U.S. Cl. CPC G16B 35/00; G16B 15/30; C12N 15/1089

(57) ABSTRACT

The present disclosure provides methods and systems for identifying insecticidal protein-encoding GIs (GIs). The present disclosure also teaches methods for producing sequenced and assembled metagenomic libraries that are amenable to GI search bioinformatic tools and techniques.

### CROSS REFERENCE TO RELATED APPLICATIONS

This application claims the benefit of priority from U.S. provisional Application No. 63/085,553, filed on Sep. 30, 2020, which is herein incorporated by reference in its entirety.

### FIELD

The present disclosure generally relates to systems and methods for insecticidal protein discovery. The disclosed systems and methods result in sequenced metagenomic databases that are amenable to in silico insecticidal protein discovery pipelines. Methods for identifying and validating new insecticidal protein-encoding Genomic Islands (GIs) are also provided.

### BACKGROUND OF THE INVENTION

Insecticidal proteins from microbes or plants are safer and environmentally sound substitutes for chemical insecticides. The target specificity of these proteins enables them to affect one specific insect and prevents them from harming animals or desirable organisms. Furthermore, since insecticidal proteins are often effective in small quantities and decompose quickly, they do not lead to pollution.

Bacillus thuringiensis (Bt) is a source of the insecticidal parasporal crystal inclusions (CPIs), which have been used for 30 years to control insects. Many plants have been genetically engineered to express CPIs. However, continuous expression of CPIs also imposes strong selection for resistance in target pest populations. Consequently, the agricultural industry has seen an alarming rate of insect populations becoming resistant to Bt crops. Furthermore, Bt proteins have a limited range of activity and are not effective against some of the currently problematic insect species.

Thus, there is an ongoing and unmet need for methods, systems, and tools to identify new insecticidal proteins.

### SUMMARY OF THE INVENTION

The present disclosure provides novel methods and systems for identifying new insecticidal proteins from a digital metagenomics library and methods of developing a digital metagenomics library. The present invention, is based, in part, on the inventor's discovery that genes encoding insecticidal proteins are enriched in genomic islands (GIs) located within microbial genomes. In some embodiments, the GIs comprise between 10 kilo-base pair (kb) and 200 kb regions of DNA. In some embodiments, GIs are identified in long assemblies of genomic DNA within the digital metagenomics library. Traditional methods of generating a digital metagenomics libraries do not yield genomic DNA assemblies of sufficient size to identify GIs. For example, the average DNA assembly produced by shotgun sequencing, a traditional method of generating a metagenomics library, results in an average DNA assembly comprising 2 kb. In some embodiments, the present disclosure provides methods of identifying insecticidal proteins from a digital metagenomics library comprising DNA assemblies with average lengths that are larger than 5kb or 10 kb.

In some embodiments, the present disclosure provides an in silico method for identifying a candidate insecticidal protein from a digital metagenomics library, said method comprising the steps of:
(a) querying a digital metagenomics library from a microbial population for a signal indicative of a genomic island (GI);
(b) supplying the output of said query as a plurality of GI-associated digital feature sets comprising all gene open reading frames within a predetermined threshold parameter from the signal indicative of the GI;
(c) screening the GI-associated digital feature sets for additional indications of an insecticidal protein encoding gene; and
(d) selecting one or more insecticidal protein encoding gene(s) from among the GI-associated digital feature sets screened in step (c), thereby identifying a candidate insecticidal protein from the digital metagenomics library.

In some embodiments, the additional indications of an insecticidal protein encoding gene comprise the presence of a pathogenicity factor within the open reading frame of the GI-associated digital feature sets, said pathogenicity factor selected from the group consisting of: predicted molecular weight, predicted hydrophobicity, presence or lack of transmembrane domain, presence or lack of other domains of interest (e.g. DUF domain), sequence similarity to known insecticidal proteins, sequence similarity to other proteins found in GIs, and predicted taxonomy.

In some embodiments, the step of screening of step (c) is conducted by experimentally confirming insecticidal function for a protein encoded by the open reading frame within the GI-associated digital feature sets.

In some embodiments, experimental confirmation of insecticidal function is conducted by expressing the protein encoded by the open reading frame within the GI-associated digital features sets in a microbial culture, and exposing said microbial culture to an insect.

In some embodiments, the predetermined threshold parameter is less 5 kb, 6 kb, 7 kb, 8 kb, 9 kb, or 10 kb from the signal indicative of the GI.

In some embodiments, the signal indicative of a GI comprises the presence of two or more GI indicators, selected from the group consisting of: a gene encoding an integrase, a gene encoding a transposases, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage and a phage gene.

In some embodiments, the GI-associated digital feature sets comprise 3, 4, 5, 6, 7, 8, 9, or more GI indicators.

In some embodiments, the digital metagenomics library comprises: sequenced and digitally assembled contig sequences having an average length of at least about 10 kb, 15 kb, 20 kb, 30 kb, 35 kb, or 40 kb.

In some embodiments, the digital metagenomics library comprises: sequenced and digitally assembled contig sequences having an N50 length of at least about 10 kb, 15 kb, 20 kb, 30 kb, 35 kb, or 40 kb.

In some embodiments, the entire digitally metagenomics library is at least about 50 MB, 100 MB, 200 MB, 300 MB, 400 MB, or 500 MB in size.

In some embodiments, the digital metagenomics library comprises: sequenced and digitally assembled contig sequences having an average length of at least about 10 kb or 20 kb and the entire digitally assembled library is at least about 500 MB in size.

In some embodiments, the digital metagenomics library comprises: sequenced and digitally assembled contig sequences having an average length of at least about 10 kb or 20 kb and the entire digitally assembled library is at least about 1 TB in size.

In some embodiments, the digital metagenomics library comprises: sequenced and digitally assembled contig sequences having an N50 length of at least about 10 kb or 20 kb and the entire digitally assembled library is at least about 1 TB in size.

In some embodiments, the digital metagenomics library comprises: sequenced and digitally assembled sequences having an average length of at least about 10 kb or 20 kb and the entire digitally assembled library is about 500 MB to about 1 TB in size.

In some embodiments, at least one of the GI indicators is identified using an HMM model trained on known sequences comprising the GI indicator.

In some embodiments, at least one of the pathogenicity factor(s) is identified using an HMM model trained on known sequences comprising the pathogenicity factor.

In some embodiments, the method further comprises the step of: (e) expressing the candidate insecticidal protein in a microbial culture, and exposing the expressed candidate insecticidal protein to an insect.

In some embodiments, the method further comprises the step of: (e) expressing the candidate insecticidal protein in a microbial culture, and exposing said microbial culture to an insect.

In some embodiments, the method further comprises the step of: (e) expressing the candidate insecticidal protein in a microbial culture, and exposing the expressed candidate insecticidal protein to an insect, wherein the insect is exposed to the microbial culture expressing the candidate insecticidal protein.

In some embodiments, the method further comprises the step of: (e) expressing the candidate insecticidal protein in a microbial culture, and exposing the expressed candidate insecticidal protein to an insect, wherein the insect is exposed to the microbial culture expressing the candidate insecticidal protein, and wherein the microbial culture is lysed before the insect is exposed to it.

In some embodiments, the expressed candidate insecticidal protein inhibits the growth or development of the insect. In some embodiments, the expressed candidate insecticidal protein kills the insect.

In some embodiments, the candidate insecticidal protein is part of a multi-gene toxin complex.

In some embodiments, the present disclosure provides a method for assembling a deeply sequenced long DNA contig metagenomic library, comprising:
(a) providing an unsequenced and unassembled metagenomic DNA sample comprising whole genomes;
(b) reducing the genomic complexity of the metagenomic DNA sample by:
   (i) cloning DNA fragments from the metagenomic library into a plurality of vectors to create a metagenomic vector fragment library that comprises the DNA from the unsequenced and unassembled metagenomic DNA sample;
   (ii) pooling the vectors from the metagenomic vector fragment library into a plurality of discrete mini-metagenome subunits that comprise from about 1,000 to about 20,000 pooled vectors each, to create a mini-metagenome library that comprises within the plurality of mini-metagenome subunits the DNA from the unsequenced and unassembled metagenomic DNA sample;
(c) performing intra-pool sequencing and assembly of the metagenomic DNA contained in the pooled vectors present in the plurality of discrete mini-metagenome subunits of the mini-metagenome library to create sequenced and assembled DNA contigs; wherein the average sequenced and assembled DNA contig length is at least about 10 kb, thereby creating a sequenced and assembled intermediary DNA contig length mini-metagenome library; and
(d) optionally performing inter-pool DNA contig assembly, by further assembling a plurality of sequenced and assembled DNA contigs from the intermediary DNA contig length mini-metagenome library to create a long DNA contig length metagenomic library.

In some embodiments, the unsequenced and unassembled metagenomic DNA sample comprises at least about 50, 100, 500, 1000, or 10000, different whole genomes.

In some embodiments, the unweighted average size of the different whole genomes in the unsequenced and unassembled metagenomic DNA sample is at least about 1 MB, 2 MB, 3 MB, 4 MB, or 5 MB.

In some embodiments, the long DNA contig length metagenomic library comprises a plurality of sequenced and assembled DNA contigs with a length of at least about 10 kb, 15 kb, 20 kb, 25 kb, 30 kb, 35 kb, 40 kb, 45 kb, 50 kb, or 100 kb.

In some embodiments, the method further comprises arraying the intermediary DNA contig length mini-metagenome library.

In some embodiments, the method further comprises arraying the long DNA contig length metagenomic library.

In some embodiments, the method further comprises arraying the intermediary DNA contig length mini-metagenome library, or the long DNA contig length metagenomic library, in a bacterial cell or DNA form.

In some embodiments, the method further comprises arraying the plurality of discrete mini-metagenome subunits into a real coordinate space and assigning identifiers to each subunit.

In some embodiments, the method further comprises: arraying the plurality of discrete mini-metagenome subunits into a multi-well microtiter plate.

In some embodiments, the method further comprises: arraying the plurality of discrete mini-metagenome subunits into a 96-well microtiter plate

In some embodiments, the vectors comprise plasmids.

In some embodiments, the vectors comprise cosmids, fosmids, BACs, YACs, or a combination thereof.

In some embodiments, the vectors comprise cosmids.

In some embodiments, the metagenomic vector fragment library in step b) comprises at least about 1 M or 10 M vectors.

In some embodiments, the vectors comprise cosmids, and the metagenomic vector fragment library in step (b) comprises at least about 10 M cosmids.

In some embodiments, the vectors comprise cosmids, and the metagenomic vector fragment library in step (b) comprises at least about 20 M cosmids.

In some embodiments, step (b) of the method comprises cloning DNA fragments of less than about 200 kb, from the metagenomic library into a plurality of vectors.

In some embodiments, step (b) of the method comprises cloning DNA fragments of less than about 100 kb, from the metagenomic library into a plurality of vectors.

In some embodiments, step (b) of the method comprises cloning DNA fragments of less than about 50 kb, from the metagenomic library into a plurality of vectors.

In some embodiments, step (b) of the method comprises cloning DNA fragments of about 20 kb to about 50 kb, from the metagenomic library into a plurality of vectors.

In some embodiments, step (b) of the method comprises cloning DNA fragments of about 30 kb to about 45 kb, from the metagenomic library into a plurality of cosmids.

In some embodiments, the discrete mini-metagenome subunits in step (b) comprise from about 3,000 to about 15,000 pooled vectors each.

In some embodiments, the discrete mini-metagenome subunits in step (b) comprise from about 5,000 to about 12,000 pooled cosmid vectors each.

In some embodiments, the sequenced and assembled intermediary DNA contig length mini-metagenome library comprises contigs with an average length of at least about 10 kb, 15 kb, 20 kb, 25 kb, or 30 kb.

In some embodiments, step (c) of the method comprises simultaneously assembling all the DNA contigs contained in the pooled vectors present in an individual discrete mini-metagenome subunit from the plurality.

In some embodiments, intra-pool sequencing of step (c) is performed utilizing single molecule sequencing.

In some embodiments, intra-pool sequencing of step (c) is performed utilizing sequencing by synthesis (SBS).

In some embodiments, intra-pool sequencing of step (c) is performed utilizing single molecule, real-time (SNRT) sequencing.

In some embodiments, intra-pool sequencing of step (c) is performed utilizing nanopore sequencing.

In some embodiments, the discrete mini-metagenome subunits in step (b) comprise from about 5,000 to about 12,000 pooled cosmid vectors each, and comprising in step (c): simultaneously assembling all the DNA contigs contained in the pooled vectors present in an individual discrete mini-metagenome subunit from the plurality.

### DESCRIPTION OF THE DRAWINGS

FIG. 1 depicts the steps of the insecticidal discovery platform of the present disclosure.

FIG. 2 depicts a diagram of DNA sequencing multiplexing strategies using barcodes. Distinguishable sequences can be added to DNA prior to sequencing (e.g., through the addition of adaptor sequences). DNA fragments with different barcodes can then be pooled (i.e., multiplexed) into a single sequence run. The barcodes are identified in post-sequencing processing, and are used to separate reads belonging to the different DNA samples (i.e., demultiplexing).

FIG. 3 depicts initial steps of the library preparation methods of the present disclosure. DNA extracted from an environmental sample is cloned into a cosmid backbone, packaged via phage, and transduced into an E. coli host to create a metagenomics DNA library.

FIG. 4 depicts of steps of the library preparation methods of the present disclosure. E. coli containing cosmids (one per cell) from the metagenomic DNA library are silo pooled into mini-meta genomes prior to sequencing

FIG. 5 depicts assembly steps of the digital metagenomic library of the present disclosure. A two phased assembly method is used to obtain longer assemblies.

FIG. 6 depicts arraying of mini-metagenomes pools as either E. coli or DNA to create physical metagenomic libraries. These arrayed libraries are later used to recover sequences of interest for further analysis.

FIG. 7 illustrates the presence of genes encoding insecticidal proteins located near signals indicative of a genomic island.

FIG. 8 shows that long assemblies within a Digital Metagenomics Library (DML) facilitates the detection of insecticidal proteins, which cluster within the genome.

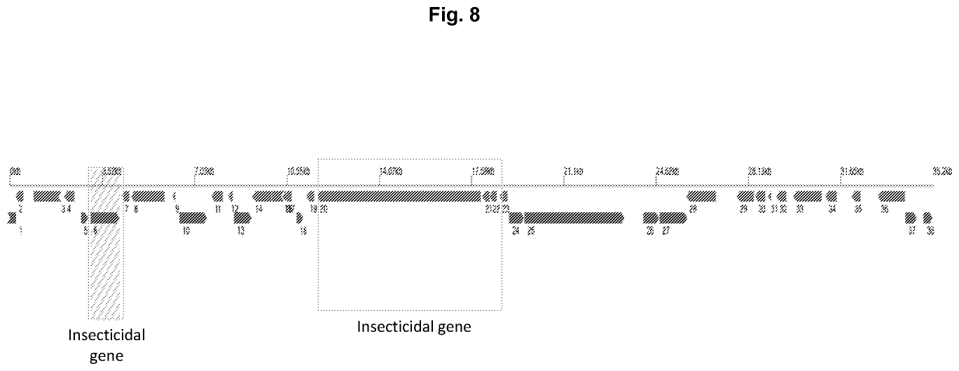

FIG. 9 shows that DMLs containing long assemblies are necessary to detect multi-gene toxin complexes, e.g., candidate insecticidals, that have a large sequence length.

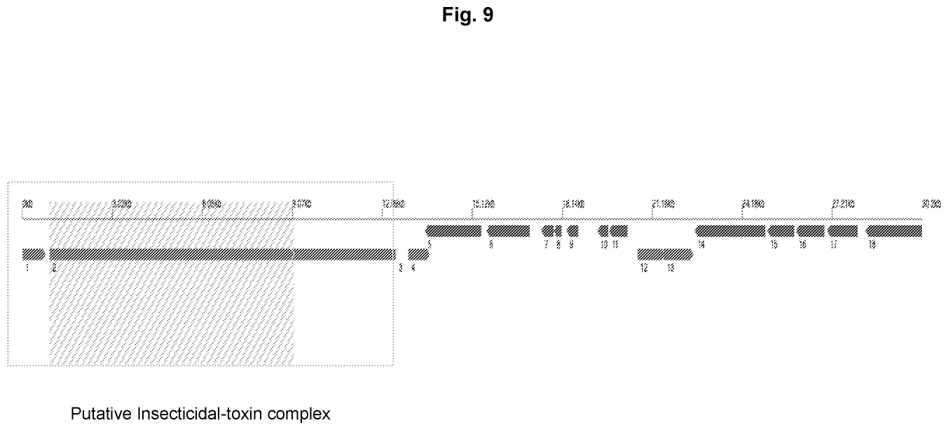

### DETAILED DESCRIPTION OF THE INVENTION

### Definitions

As used herein, the verb “comprise” is used in this description and in the claims and its conjugations are used in its non-limiting sense to mean that items following the word are included, but items not specifically mentioned are not excluded.

As used herein, the term “about” refers to plus or minus 10% of the referenced number unless otherwise stated or otherwise evident by the context, and except where such a range would exceed 100% of a possible value, or fall below 0% of a possible value, such as less than 0% content of an ingredient, or more than 100% of the total contents of a composition. For example, reference to a distance of “about 10 kb” means a distance between 9 kb and 11 kb.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

This disclosure refers to a part, such as a protein, as being “engineered” into a host cell when the genome of the host cell is modified (e.g., via insertion, deletion, replacement of genes, including insertion of a plasmid encoding for an insecticidal protein or an entire GI) so that the host cell produces at least one new gene/protein (e.g., an insecticidal).

As used herein, the “confidence score” is a measure of the confidence assigned to a classification or classifier. For example, a confidence score may be assigned to the identification of an amino acid sequence as encoding an insecticidal gene. Confidence scores include bit scores and e-values, among other. A “bit score” provides the confidence in the accuracy of a prediction. “Bits” refers to information content, and a bit score generally indicates the amount of information in the hit. A higher bit score indicates a better prediction, while a low score indicates lower information content, e.g., a lower complexity match or worse prediction. An “e-value” as used herein refers to a measure of significance assigned to a result, e.g., the identification of a sequence in a database predicted to encode a protein having the same function as the search protein (e.g., an insecticidal protein). An e-value generally estimates the likelihood of observing a similar result within the same database. The lower the e-value, the more significant the result is.

A “Hidden Markov Model” or “HMM” as used herein refers to a statistical model in which the system being modeled is assumed to be a Markov process with unobservable (i.e. hidden) states. As applied to amino acid sequences, an HMM provides a way to mathematically represent a family of sequences. It captures the properties that sequences are ordered and that amino acids are more conserved at some positions than others. Once an HMM is constructed for a family of sequences, new sequences can be scored against it to evaluate how well they match and how likely they are to be a member of the family.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by Clustal Omega® using default parameters.

As used herein, a residue (such as a nucleic acid residue or an amino acid residue) in sequence “X” is referred to as corresponding to a position or residue (such as a nucleic acid residue or an amino acid residue) “a” in a different sequence “Y” when the residue in sequence “X” is at the counterpart position of “a” in sequence “Y” when sequences X and Y are aligned using amino acid sequence alignment tools known in the art, such as, for example, Clustal Omega or BLAST®.

When percentage of sequence identity is used in reference to proteins it is recognized that residue positions which are not identical can differ by conservative amino acid substitutions, where amino acid residues are substituted for other amino acid residues with similar chemical properties (e.g., charge or hydrophobicity) and therefore do not change the functional properties of the molecule. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). Similarity can be a more sensitive measure of relatedness between sequences than identity; it takes into account not only identical (i.e. 100% conserved) residues but also non-identical yet similar (in size, charge, etc.) residues. In some embodiments, % similarity as used herein is based on the BLOSUM90 substitution matrix.

The methods and systems of the present disclosure can be used to identify sequences that are homologous/orthologous to one or more target genes/proteins, such as insecticidal proteins, signals indicative of a PAI, and additional indications of an insecticidal protein encoding gene. As used herein, homologous sequences are sequences (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between), and (b) carry out the same or similar biological function.

In some embodiments, the present disclosure teaches methods and systems for identifying homolog or ortholog of a target protein or gene. As used herein in the terms “target protein” or “target gene” refers to a starting gene or protein (e.g., nucleic acid or amino acid sequence) for which homologs or orthologs are sought.

As used herein, the term “ortholog” refers to a nucleic acid or protein that is homologous to a target sequence, and from different species. As used herein, the term “distantly related orthologs” refers to an ortholog that: (a) shares less than 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% sequence identity with a target protein/gene (including all ranges and subranges therebetween), while (b) performing the same function as the target protein/gene.

The present disclosure teaches methods and systems for identifying homologs and orthologs of target genes/proteins, wherein said homologs and orthologs perform the same function as the target gene/protein. As used herein, the term “same function” refers to interchangeable genes or proteins, such that the newly identified homolog or ortholog can replace the original target gene/protein while maintaining at least some level of functionality. In some embodiments, an enzyme capable of catalyzing the same reaction as the target enzyme will be considered to perform the same function. In some embodiments, a transcription factor capable of regulating the same gene as the target transcription factor will be considered to perform the same function. In some embodiments, a small RNA capable of complexing with the same (or equivalent) nucleic acid as the target small RNA will be considered to perform the same function.

Performing the “same function” however, does not necessarily require the newly identified homolog or ortholog to perform all of the functions of the target gene/protein, nor does it preclude the newly identified homolog from being able to perform additional functions beyond those of the target gene/protein. Thus, in some embodiments, a newly identified homolog or ortholog may have, for example, a smaller pool of usable reactants, or may produce additional products, when compared to the target enzyme.

Persons having skill in the art will also understand that the term “the same function” may, in some embodiments, also encompass congruent, but not identical functions. For example, in some embodiments, a homolog or ortholog identified though the methods and systems of the present disclosure may perform the same function in one organism, but not be capable of performing the same function in another organism. One illustrative example of this scenario is an ortholog subunit of a multi-subunit enzyme, which is capable of performing the same function when expressed with other compatible subunits of one organism, but not be directly combinable with subunits from different organisms. Such a subunit would still be considered to perform the “same function.” Techniques for determining whether an identified gene/protein performs the same function as the target gene/product are discussed in detail in the present disclosure.

The term “polypeptide” or “protein” or “peptide” is specifically intended to cover naturally occurring proteins, as well as those which are recombinantly or synthetically produced. It should be noted that the term “polypeptide” or “protein” may include naturally occurring modified forms of the proteins, such as glycosylated forms. The terms “polypeptide” or “protein” or “peptide” as used herein are intended to encompass any amino acid sequence and include modified sequences such as glycoproteins.

The term “prediction” is used herein to refer to the likelihood, probability or score that a protein will perform a given function, or to which a series of genes form a GI encoding an insecticidal protein.

In the description, the term “open reading frame” or an ORF refers to a DNA sequence encoding a protein gene, said open reading frame ranging from a translation start codon (e.g., ATG) to a stop codon (e.g., TGA, TAA, TAG). For the purposes of this application, DNA sequences which are either computationally predicted (or empirically determined) not to produce a protein are not considered to comprise ORFs. In addition, an ORF without an associated transcription start site (i.e., a DNA sequence that would not be transcribed to mRNA) would not be considered an ORF. Additionally, ORFs encoding for less than 10, 20, 30, 40 , 50, 60, 70, 80, 90, or 100 amino acids, are not considered ORFs for the purposes of the proximity calculations between computationally determined PAI-associated digital feature set.

The terms “training data”, “training set” or “training data set” refers to a data set for which a classification (e.g., enzyme function) may be known. In some embodiments, training sets comprise input and output variables and can be used to train the model. The values of the features for a set can form an input vector, e.g., a training vector for a training set. Each element of a training vector (or other input vector) can correspond to a feature that includes one or more variables. For example, an element of a training vector can correspond to a matrix. The value of the label of a set can form a vector that contains strings, numbers, bytecode, or any collection of the aforementioned datatypes in any size, dimension, or combination. In some embodiments, the “training data” is used to develop a machine learning predictive model capable of identifying other sequences likely to exhibit the same function as a target gene/protein. In some embodiments, the training data set includes a genetic sequence input variable with one or more genetic sequences (e.g., nucleotides or amino acids) encoding proteins capable of performing the same function as the target protein. In some embodiments, the training data set can also contain sequences that are labeled as not performing the same function.

In some embodiments, the training data set also includes a “phenotypic performance output variable”. In some embodiments, the “phenotypic output variable” can be binary (e.g., indicating whether an associated sequence exhibits the same function or not). In some embodiments, the phenotypic output variable can indicate a level of certainty about a stated function, such as indicating whether same function has been experimentally validated as positive or negative, or is predicted based on one or more other factors. In some embodiments, the phenotypic output variable is not stored as data but is merely the fact of performing a given function. For example, a training data set may comprises sequences known or predicted to perform a target function. In such embodiments, the genetic input variables are the sequences and the phenotypic performance output variables are the fact of performing the function or being predicted to perform the function. Thus, in some embodiments, inclusion in the list implies a phenotypic performance variable indicating that the sequences perform the same function.

As used herein the terms “host cell” “cellular organism”, “microorganism”, or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes.” In some embodiments, suitable microorganisms are found in lists present in the disclosure. This characterization can refer to not only the identified taxonomic genera of the aforementioned lists, but also the identified taxonomic species, as well as the various novel and newly identified or designed strains of any organism in said tables or figures. The same characterization holds true for the recitation of these terms in other parts of the Specification, such as in the Examples. In some embodiments, the microorganisms are yeast cells. Non-limiting examples of yeast cells include Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica. In some embodiments, the microorganism is an algal cell such as, Chlamydomonas (e.g., C. Reinhardtii) and Phormidium (P. sp. ATCC29409).

In some embodiments, the microorganism is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, The rmosynechococcus, Thermococcus, Ureaplasma, Xanthomonas, Xylella, Yersinia, and Zymomonas. In some embodiments, the microorganism is Corynebacterium glutamicum.

In some embodiments, the bacterial microorganism is a bacterial industrial strain. Numerous bacterial industrial strains are known and suitable in the methods and compositions described herein.

In some embodiments, the bacterial microorganism is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. finnus, B. alkaophius, B. licheniformis, B. clausii, B. stearothermophilus, B. halodurans and B. amyloliquefaciens. In some embodiments, the microorganism is an industrial Bacillus strain including but not limited to B. subtilis, B. pumilus, B. licheniformis, B. megaterium, B. clausii, B. stearothermophilus and B. amyloliquefaciens. In some embodiments, the microorganism is an industrial Clostridium species (e.g., C. acetobutylicum, C. tetani E88, C. lituseburense, C. saccharobutylicum, C. perfringens, C. beijerinckii). In some embodiments, the microorganism is an industrial Corynebacterium species (e.g., C. glutamicum, C. acetoacidophilum). In some embodiments, the microorganism is an industrial Escherichia species (e.g., E. coli). In some embodiments, the microorganism is an industrial Erwinia species (e.g., E. uredovora, E. carotovora, E. ananas, E. herbicola, E. punctata, E. terreus). In some embodiments, the microorganism is an industrial Pantoea species (e.g., P. citrea, P. agglomerans). In some embodiments, the microorganism is an industrial Pseudomonas species, (e.g., P. putida, P. aeruginosa, P. mevalonii). In some embodiments, the microorganism is an industrial Streptococcus species (e.g., S. equisimiles, S. pyogenes, S. uberis). In some embodiments, the microorganism is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the microorganism is an industrial Zymomonas species (e.g., Z. mobilis, Z. lipolytica), and the like.

In some embodiments, the present disclosure teaches a metagenomic database comprising genetic sequences of at least one, two, three, four, five, six, seven, eight, nine, ten, or more uncultured microbe or microorganism. As used herein, the term “uncultured microbe” “uncultured cell” or “uncultured organism” refers to a cell that has not been adapted to grow in the laboratory. In some embodiments the uncultured microbes/cells/organism has not been previously sequenced, or the genomic sequence is not publicly available.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic and non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell

The term “wild-type microorganism” or “wild-type host cell” describes a cell that occurs in nature, i.e. a cell that has not been genetically modified.

The term “genetically engineered” may refer to any manipulation of a host cell's genome (e.g. by insertion, deletion, mutation, or replacement of nucleic acids).

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell.

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titre” or “titer” is defined as the strength of a solution or the concentration of a substance in solution. For example, the titre of a product of interest (e.g. small molecule, peptide, synthetic compound, fuel, alcohol, etc.) in a fermentation broth is described as g of product of interest in solution per liter of fermentation broth (g/L).

A “barcode” or “molecular barcode” is a material for labeling. The barcode can label a molecule such as a nucleic acid or a polypeptide. The material for labeling is associated with information. In some embodiments, a barcode is called a sequence identifier (i.e. a sequence-based barcode or sequence index). A barcode is a particular nucleotide sequence. A barcode is used as an identifier. A barcode is a different size molecule or different ending points of the same molecule. Barcodes can include a specific sequence within the molecule and a different ending sequence. For example, a molecule that is amplified from the same primer and has 25 nucleotide positions is different than a molecule that is amplified and has 27 nucleotide positions. The addition positions in the 27mer sequence is considered a barcode. A barcode is incorporated into a polynucleotide. A barcode is incorporated into a polynucleotide by many methods. Some non-limiting methods for incorporating a barcode can include molecular biology methods. Some non-limiting examples of molecular biology methods to incorporate a barcode are through primers (e.g., tailed primer elongation), probes (i.e., elongation with ligation to a probe), or ligation (i.e., ligation of known sequence to a molecule).

The term “insecticidal protein” refers to a protein that increases mortality or inhibits the growth rate of an insect. As used herein, the term “insect” includes all organisms in the class “Insecta”. Insects may be at any form of their life cycle, including, for example, eggs, larvae, nymphs, and adults.

The term “genomic island,” abbreviated GI, refers to a nucleic acid ranging from about 5 kb to about 200 kb that comprises a cluster of genes that are thought to have been acquired by horizontal gene transfer (HGT). In some embodiments, the present disclosure teaches that GIs are identified by the presence of one or more signals indicative of a GI. In some embodiments, non-limiting examples of signals indicative of a GI include genes that encode integrases, transposases, endonucleases, adhesins, secretion systems, toxins, invasins, modulins, iron uptake systems, proteases, insecticidal proteins, virulence factors, transfer RNA (tRNA), a prophage, a phage, or a domain of unknown function. Non-limiting examples of genomic islands include pathogenicity islands (PAls), resistance islands, metabolic islands, and symbiosis islands.

### Challenges of GI Discovery in Metagenome Sequences

Compared to regular (e.g., publicly available complete) genome sequences, the analysis of metagenomic sequence data for the identification of insecticidal proteins within GIs presents several key challenges.

One challenge is the ability to generate sufficiently long portions of the genome to permit for meaningful sequence analysis and subsequent GI recovery. In post-sequencing genomic assembly, there is an expectation that a sample contains a single species (apart from any contamination, which can be screened for prior to assembly). This expectation allows assembly tools to make certain assumptions that facilitate assembly. The expected coverage of the target genome can be predicted from the total size of the data set divided by the estimated size of the genome. DNA inputs into sequencers are assumed to be relatively stable for sequences across the genome. Therefore, it can be assumed that nodes or edges in a graph occurring with very low coverage compared to the expected coverage are likely the result of sequencing errors or low level contamination, and the graph is simplified considerably by removing such nodes or paths. Similarly, nodes with much higher than average coverage can be assumed to be part of repeat structures within the genome. The typical optimal sequence coverage for a single genome assembler is in the 20-200× range, with a common ‘sweet spot’ of ˜50× (Desai A, Marwah VS, Yadav A, et al. Identification of optimum sequencing depth especially for de novo genome assembly of small genomes using next generation sequencing data. PLOS One 2013; 8(4):e60204).

However, in metagenomic data sets this assumption and simplifications cannot be made. Lower coverage nodes may originate from genomes with a lower abundance, not from errors, and so should not be discarded out of hand. Compounding this problem, the number of species within a sample, and the distribution of abundances of species is unknown. Abundance in heterogeneous samples often follows a power law, which means that many species will occur with similarly low abundances making the problem of distinguishing one from another problematic (Li D, Liu CM, Luo R, et al. MEGAHIT: an ultra-fast single-node solution for large and complex metagenomics assembly via succinct de Bruijn graph. Bioinformatics 2015; 31(10):1674-6). The low coverage of most species means de novo assembly is unlikely unless the genome in question is relatively small.

Indeed, assemblies from most complex metagenomic libraries are highly limited in length, and thus prevent meaningful GI analysis. Short assemblies often do not include complete GIs, which makes it difficult for bioinformatic algorithms to identify and analyze clusters. Because of these limitations, there have not been any in silico bioinformatics GI analysis of highly complex metagenomic libraries. Instead, most bioinformatics work reported to date has either relied on publicly available pre-assembled libraries, or limited small metagenome assemblies of less than genomes.

### Methods, Systems and Tools of the Present Insecticidal Discovery Platform

The present disclosure provides several advanced metagenomic library preparation and bioinformatics analysis pipelines that enable large quantities of GIs to be mined from microorganisms without having to culture the GI-containing microorganism. The tools provided in this disclosure thus provide an incredible opportunity to elucidate the secondary metabolism properties of microbial dark matter, which is the uncultured majority of microbial diversity.

In some embodiments, the present disclosure teaches an insecticidal protein discovery workflow comprising: 1) metagenomic library creation, 2) sequencing and creation of digital metagenomics libraries (“DML”), 3) Querying DML, and identifying GIs based on novel bioinformatics discovery approaches, 4) screening for GI-associated digital feature sets comprising insecticidal protein encoding genes and selecting insecticidal proteins. Each of the elements of the insecticidal protein discovery platform are discussed in more detail below.

### Digital Metagenomics Library Creation

In some embodiments the present disclosure teaches methods and systems for identifying GIs from metagenomic libraries. The present disclosure also teaches methods and systems for generating metagenomic libraries amenable to GI bioinformatic searching.

For the purposes of this disclosure, a metagenomic library may be defined in the following ways:
1) A physical or digital sequence library that comprises the genomes of uncultured species (e.g., a library derived from environmental samples without an intervening culturing step). In some embodiments, the uncultured species are from yeast, fungus, bacterium, archae, protist, virus, parasite or algae species. The uncultured species may be obtained from any source, e.g., soil, gut, aquatic habitat. In some embodiments, a library is considered a metagenomics library if a majority of the sequence within the assembled library is from uncultured organisms, and if the library meets other size limitations. In some embodiments, the physical and/or digital sequence library of the present disclosure is representative of the environmental sample from which it was extracted, and is not an agglomeration of existing small (e.g., less than 100 organism) assemblies. Any exogenously added/spiked sequence beyond that sourced from the environmental sample may be considered outside of the library of the present disclosure.
2) A physical or digital sequence library that meets the definition of point 1 above, and further wherein a majority of the sequence within the library is from uncultured organisms. In some embodiments, a digital metagenomics library is considered to contain a majority of sequence from uncultured organisms if it is produced by sequencing physical libraries where a majority of the organisms in the library are uncultured. In some embodiments, a digital metagenomics library is considered to contain a majority of sequence from uncultured organisms if it is produced by sequencing physical libraries where none of the organisms were cultured prior to sequencing. In some embodiments, a library is considered a metagenomics library if substantially all of the sequence within the assembled library is from uncultured organisms, and if the library meets other size limitations. As used in this context, the term “substantially all” refers to a library wherein at least 90% of the assembled sequence is from uncultured organisms. In some embodiments, a digital metagenomics library is considered to contain substantially all of its sequence from uncultured organisms if it is produced by sequencing physical libraries where substantially all of the organisms in the library are uncultured. In some embodiments, a digital metagenomics library is considered to contain substantially all of the sequence from uncultured organisms if it is produced by sequencing physical libraries where none of the organisms were cultured prior to sequencing.
3) A physical or digital sequence library that meets the definition of points 1 and/or 2 above, and further comprises more than one uncultured species' genome. In some embodiments the metagenomic library comprises the genomes of at least 100, 500, 1000, 10⁴, 10⁵, 10⁶, 10⁷ or more uncultured species. In some embodiments, the number of assembled genomes in a digital metagenomics library is calculated by dividing the total assembled sequence in the DML, and dividing it by the average size of genomes of the kind of organisms expected to be present in the genome. In some embodiments, the number of assembled genomes in a digital metagenomics library is assessed by counting the number of unique 16s rRNA sequences in the DML. In some embodiments, the number of assembled genomes in a digital metagenomics library is assessed by counting the number of unique Internal transcribed spacers (ITS) in the DML.
4) A digital sequence library that meets the definition of one or more of points 1-3 above, and wherein the digital metagenomics library is at least about 50 Mb, 60 Mb, 70 Mb, 80 Mb, 100 Mb, 110 Mb, 120 Mb, 130 Mb, 140 Mb, 150 Mb, 160 Mb, 170 Mb, 180 Mb, 190 Mb, 200 Mb, 210 Mb, 220 Mb, 230 Mb, 240 Mb, 250 Mb, 260 Mb, 270 Mb, 280 Mb, 290 Mb, 300 Mb, 310 Mb, 320 Mb, 330 Mb, 340 Mb, 350 Mb, 360 Mb, 370 Mb, 380 Mb, 390 Mb, 400 Mb, 410 Mb, 420 Mb, 430 Mb, 440 Mb, 450 Mb, 460 Mb, 470 Mb, 480 Mb, 490 Mb, 500 Mb, 550 Mb, 600 Mb, 650 Mb, 700 Mb, 750 Mb, 800 Mb, 850 Mb, 900 Mb, 950 Mb, 1000 Mb, 1050 Mb, 1100 Mb, 1150 Mb, 1200 Mb, 1250 Mb, 1300 Mb, 1350 Mb, or 1400 Mb in size. Assembled sequence is the additive lengths of all contigs in the DML.
5) A digital sequence library that meets the definition of one or more of points 1-4 above, and further comprises an N50 of at least about 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, or 35 kb (i.e., long-assembly digital metagenomic library).
6) A sequence library that meets definition of one or more of points 1-5 above, and is further distinguished from having been based on genetic material recovered directly from environmental material comprising samples of all genes from all the members of the material. “Environmental material” may include but is not limited to environmental DNA (eDNA).
7) A sequence library that meets definition of one or more of points 1-6 above, and further comprises assembled sequence reads averaging at least about 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 31 kb, 32 kb, 33 kb, 34 kb, or 35 kb.

In some embodiments, metagenomics involves the direct extraction of DNA from environmental samples. Another advantage of metagenomic databases is that they can be enriched for organisms that are more likely to comprise genes likely to encode the desired insecticidal protein. For example, GIs containing insecticidal proteins may be enriched in metagenomic databases produced from microbial samples that have been collected from soil containing plants where insects of interest reside. GIs containing insecticidal proteins may also be enriched for by pretreating microbial samples with heat. Thus, the methods and systems of the present disclosure benefit from the wide diversity of sequences available through metagenomic databases, and from the potential for enriching such databases for the desired end use.

Microorganisms play an essential role in the function of ecosystems and are well represented quantitatively. Environmental samples, such as soil samples, food samples, or biological tissue samples can contain extremely large numbers of organisms and, consequently, generate a large set of genomic data. For example, it is estimated that the human body, which relies upon bacteria for modulation of digestive, endocrine, and immune functions, can contain up to 100 trillion organisms. In addition, it is estimated that one gram of soil can contain between 1,000 and 10,000 different species of bacteria with between 10⁷ and 10⁹ cells, including cultivatable and non-cultivatable bacteria. Reproducing this whole diversity in metagenomic DNA libraries requires the ability to generate and manage a large number of clones. In some embodiments, the metagenomic database may comprise at least one, several dozen, hundreds of thousands, or even several million recombinant clones which differ from one another by the DNA which they have incorporated. In some embodiments, the metagenomic library may be constructed from metagenomic fragments and/or assembled into contigs, as described in U.S. Pat. Nos 8,478,544, 10,227,585, and 9,372,959, each incorporated by reference in its entirety herein. In some embodiments, the metagenomic sequences may be assembled into whole genomes. In some embodiments, the metagenomic library may be optimized to comprise an average size (or N50) of the cloned metagenomic inserts to facilitate the search for microbial biosynthesis pathways, because these pathways are often organized in clusters in the microorganism's genome. Larger cloned fragments of DNA (e.g., larger than 10, 20 or 30 Kb), have higher chances of comprising complete GIs, which facilitates searching for insecticidal proteins.

Persons having skill in the art will be aware of the relationship between DNA, RNA, and protein sequences, and will thus be able to readily convert DNA sequence data to create metagenomic libraries with RNA or protein information. In some embodiments, the metagenomic libraries of the present disclosure comprise DNA sequences obtained from cellular populations. Thus, in some embodiments, metagenomic libraries comprise information obtained from direct DNA sequencing. In some embodiments, the metagenomic libraries comprise transcribed RNAs that are either directly measured, or predicted based on DNA sequence. Thus, in some embodiments metagenomic libraries can be searched for siRNAs, miRNAs, rRNAs, and aptamers. In some embodiments, metagenomic libraries comprise amino acid protein sequence data, either measured, or predicted based on measured DNA sequences. For example, metagenomic libraries may comprise a list of predicted or validated protein sequences that are accessible to the machine learning models described in the present disclosure.

In some embodiments, the GI discovery systems and methods of the present disclosure are applied to assembled sequence libraries from environmental samples. (“environmental libraries” or “ELs”). In some embodiments Els are deeply (i.e. at least 10× coverage) sequenced assemblies of environmental DNA samples, which have either been directly sequenced (and may thus be metagenomic samples as described above), or which have undergone at least one culturing step (e.g., to enrich for one or more kinds of organisms). In some embodiments, the ELs of the present disclosure will comprise the following properties, which improve their functioning with the MGC discovery methods and systems of the present disclosure:
1) ELs comprise a digitally assembled sequence library that is at least about 50 Mb, 70 Mb, 80 Mb, 90 Mb, 100 Mb, 110 Mb, 120 Mb, 130 Mb, 140 Mb, 150 Mb, 160 Mb, 170 Mb, 180 Mb, 190 Mb, 200 Mb, 210 Mb, 220 Mb, 230 Mb, 240 Mb, 250 Mb, 260 Mb, 270 Mb, 280 Mb, 290 Mb, 300 Mb, 310 Mb, 320 Mb, 330 Mb, 340 Mb, 350 Mb, 360 Mb, 370 Mb, 380 Mb, 390 Mb, 400 Mb, 410 Mb, 420 Mb, 430 Mb, 440 Mb, 450 Mb, 460 Mb, 470 Mb, 480 Mb, 490 Mb, 500 Mb in size. Assembled sequence is the additive lengths of all contigs in the El.
2) ELs meet the definition of points EL point 1 above, and further comprise an N50 of at least about 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb. (i.e., a long-assembly digital enviromental library)

Subsequent sections of this document teach methods of preparing the environmental libraries and metagenomic libraries used in the methods of the present disclosures. Methods discussed below for preparing metagenomic libraries also apply to environmental libraries. For example, in some embodiments, the environmental libraries of the present disclosure are still extracted from environmental samples, are siloed into pools prior to sequencing, and can optionally be assembled in two stages, as discussed below. Moreover, all of the digital searching worflows discussed in this document can also be applied to Els. That is, all references to use of the DML in methods discussed in this specification below, can be replaced with the term EL. This paragraph merely notes the applicability of the presently disclosed methods to libraries that may contain cultured organisms, but does not contradict the benefits of true metagenomic libraries, as defined above.

### Metagenomic Library Creation—DNA Extraction

The first step in producing a metagenomic library is extracting DNA from the metagenomic sample of interest (e.g., soil, river water, gut feces). Persons having skill in the art will be familiar with methods of DNA extraction. There are many commercial DNA extraction kits that are optimized for sequencing applications from metagenomic samples. MP Biomedicals for example, sells an FastDNATM Spin kit for DNA extraction from soil samples. Other known techniques are disclosed in the art (Shamim K, Sharma J, Dubey S K. Rapid and efficient method to extract metagenomic DNA from estuarine sediments. 3 Biotech. 2017; 7(3):182; see also Bag, S., Saha, B., Mehta, O. et al. An Improved Method for High Quality Metagenomics DNA Extraction from Human and Environmental Samples. Sci Rep 6, 26775 (2016); and Ahmadi, E., Kowsari, M., Azadfar, D. et al. Annals of Forest Science (2018) 75: 43).

In some embodiments, the present disclosure teaches a protocol for soil metagenomic DNA extraction comprising: a) removing non-soil debris from a soil sample with a wire mesh; b) extracting DNA from the resulting soil by adding 300mL of a CTAB-based lysis buffer (100mM Tris-HCl, 100 mM EDTA, 1.5 M NaCl, 1% (w/v) CTAB, 2% (w/v) SDS, pH 8.0), followed by incubation at 70 for 2 h with consistent inversion to mix; c) centrifuging the sample at 4,000 g for 20 minutes at 4° C. and transferring the supernatant to a clean bottle before centifuging a second time at 4,000g for 20 min. at 4° C.; d) transferring the lysate to a new bottle and adding 0.7 volumes of isopropanol and gently mixing for 30 min; e) pelting the precipitated DNA with two rounds of centrifugation at 4,000 g for 30 min. at 4° C., washing with 70% ethanol between the first and second centrifugation; f) removing the supernatant and allowing the pellet to dry; and g) resuspending the pellet in 10 mL of TE buffer. The extracted DNA can optionally be quantified by a spectrophotometer, and saved for further processing.

In some embodiments, the extracted metagenomics DNA sample comprises at least about 50 whole, distinct, genomes, for example, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, or about 10000 whole genomes. In some embodiments, the extracted metagenomics DNA sample comprises at least about 50, 100, 500, 1000, or 10,000 whole genomes.

In some embodiments, the unweighted average size of the different whole genomes in the extracted DNA sample is at least about 1 MB, 2 MB, 3 MB, 4 MB, or 5 MB. The unweighted average takes into account unique genomes, e.g., one of each unique genome in the extracted DNA sample is utilized to compute the average.

### Metagenomic Library Creation—Size Selection and Cosmid Packaging

The next step in producing a metagenomic library is cloning large fragments of the extracted DNA into a recombinant DNA vector and transducing the resulting recombinant plasmid into a microbial host for storage and propagation. In addition, the cloned DNA can be used to prepare the extracted DNA for sequencing. Persons having skill in the art will be familiar with the many methods for processing DNA for various next generation sequencing platforms. In some embodiments, however, the present disclosure teaches specific methods of pooling DNA samples to reduce the complexity of downstream genome assemblies.

In some embodiments, DNA samples are cloned into cosmid vector backbones, packaged by phage, and transduced into E. coli cells to amplify and create physical copies of extracted DNA. In some embodiments, DNA extracted from metagenomic samples is loaded and run through agarose gels for an initial size fractionation step. In some embodiments, DNA that is around 35-45 kb is excised and electroluted from the agarose gel for further processing. In some embodiments no size fraction is necessary, particularly if the phage packaging technique selectively packages inserts of the desired size (e.g., by using Gigapack III XL™ by Agilent®).

In some embodiments, the DNA is then packaged into cosmids in phages for amplification. In some embodiments, packaging DNA into cosmids comprises the following general steps: (1) ligation of the foreign DNA between two cos sites; (2) making a concatemeric DNA; (3) in vitro packaging to introduce the DNA into the phage head to form the matured phage particle; and (4) introduction of the cloned DNA into E. coli by transduction. Persons having skill in the art will be familiar with various cosmid production and amplification techniques. A non-limiting list of commercial kits for phage packaging include: MaxPlax™ Lambda Packaging Extracts Kit, Gigapack III Gold™, Gigapack III Plus™, Gigapack III XL™, Packagene®.

In some embodiments, the present disclosure teaches a protocol for lambda phage packaging, said protocol comprising the steps of: a) processing the extracted DNA with an End-It DNA End-Repair kit (Lucigen, ER0720) to produce blunt ended DNA, b) ligating 250ng of the resulting blunt-ended DNA into 500ng of a blunt-ended cosmid vector using T4 ligase, and c) packaging the resulting cosmids into phages using a MaxPlaxTM packaging kit following manufacturer's instructions.

### Metagenomic Library Creation—Silo Pooling

As discussed above, the primary challenge in applying bioinformatic tools to sequenced metagenomic libraries is the inability to assemble long sequences from complex environmental DNA samples. The present disclosure teaches complexity-reducing methods in the library preparation and assembly steps that solve the issues of the prior art, and produce digital metagenomics libraries amenable to in silico multi-gene cluster discovery.

It is not uncommon for next generation sequencing protocols to include a sample pooling step. The pooling of samples before sequencing is typically done to reduce costs, and to make efficient use of sequencers, which are often capable of sequencing much more than a single sample. The average size of a bacterial genome, for example, is about 3.65 Mb (see diCenzo G C, Finan T M. 2017. The divided bacterial genome: structure, function, and evolution. Microbiol Mol Biol Rev 81:e00019-17). Illumina's NovaSeq 6000™ sequencing machine, on the other hand, is capable of sequencing between 32 and 40 billion bases per run (i.e., roughly equivalent to about 10,000× the average bacterial genome). This type of intentional sample pooling relies on the use of barcoding technology, which allows the computer to sort the resulting sequences into files corresponding to each individual (pre-mixed) sample before genomic assembly begins.

Metagenomic DNA samples represent massive, involuntary, and unmarked, DNA pools comprising the genomes of hundreds to millions of microbes present in the original material sample (e.g., soil). Because the genomes were premixed, the resulting sequences from a metagenomic NGS must be assembled without the ability to pre-sort the reads according to which organism they belong to.

In some embodiments, the present disclosure teaches methods of silo-pooling of metagenomic samples to decrease complexity, and improve assemblies. In some embodiments, DNA cosmids from a metagenomic DNA sample are processed and stored within an E. coli library. Each colony within the E. coli library comprises one cosmid of about 35-40 kb in length. In some embodiments, breaking up the genomes of the metagenomic library into individual cosmids reduces the assembly difficulty of such fragments. This is contrasted with some traditional approaches of sequencing a whole genome at once, without first separating the library into individual cosmids.

Many traditional sequencing protocols teach extracting metagenomic environmental DNA composed of whole genomes into a single sample for shotgun sequencing (e.g., combining all clones within a metagenomic library into a single pool). The presently disclosed approach differs from these traditional approaches in that it produces a plurality of small pools of sizes that maximize use of the sequencer while still producing assemblies of sufficient quality for GI discovery.

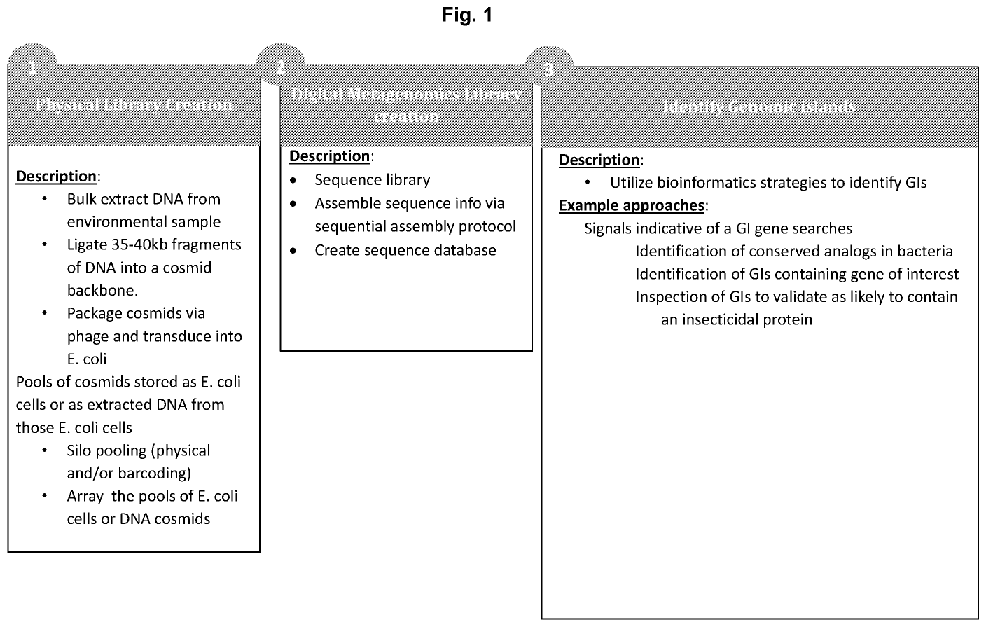

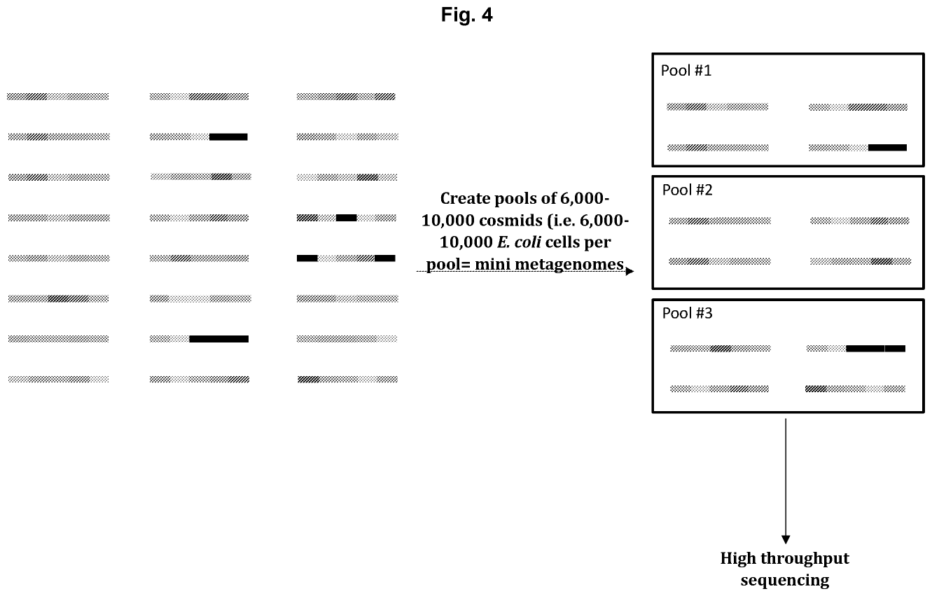

Specifically, in some embodiments, the presently disclosed methods teach the 1) cloning of fragments of genomes into cosmids with 2) selective pooling of limited numbers of E. coli colonies containing cosmids into a plurality of sequencing silos. (See step 1 of FIG. 1, and FIG. 4) The resulting sequencing silos comprise a limited number of full length cosmids, thus reducing the complexity of subsequent assemblies. As will be discussed in more detail below, the silo pooling methods reduce the problem from one of assembling, in parallel, whole genomes or 20 million cosmids corresponding to hundreds/thousands of genomes, to one in which the assembly focuses on only a few thousand cosmids.

Some publications have previously disclosed pooling of small numbers of clones, as alternatives to barcoding or whole genome sequencing (Dz\hat{ }unkova\acute{ } M, D'Auria G, Pe\acute{ }rez-Villarroya D, Moya A (2012) Hybrid Sequencing Approach Applied to Human Fecal Metagenomic Clone Libraries Revealed Clones with Potential Biotechnological Applications. PLoS One 7: e47654. ; Wang L, Hatem A, Catalyurek U V, Morrison M, Yu Z (2013) Metagenomic Insights into the Carbohydrate-Active Enzymes Carried by the Microorganisms Adhering to Solid Digesta in the Rumen of Cows. PloS One 8: e78507).). Lam et al. 2013, for example, disclosed the pooling of 92 distinct clones derived from environmental samples. (Lam KN, Hall MW, Engel K, Vey G, Cheng J, et al. (2014) Evaluation of a Pooled Strategy for High-Throughput Sequencing of Cosmid Clones from Metagenomic Libraries. PloS ONE 9(6): e98968. Doi:10.1371/journal.pone.0098968). The experiments in Lam et al. however were limited to a small number of pre-screened clones, which were sequenced to approximately 900-fold read depth and >100 fold coverage. Despite this extraordinarily high level of sequencing, Lam reported only recovering reference contigs for 77 out of the 92 original clones. The results of Lam et al. thus did not provide any expectation of success of producing digital metagenomic libraries from silo pooling of 3,000 to 14,000 cosmids, as presently disclosed.

In some embodiments, each of the resulting sequencing silos comprises between 3,000-14,000 cosmids. In some embodiments, each sequencing silo comprises 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 10,100, 10,200, 10,400, 10,500, 10,600, 10,700, 10,800, 10,900, 11,000, 11,100, 11,200, 11,300, 11,400, 11,500, 11,600, 11,700, 11,800, 11,900, 12,000, 12,100, 12,200, 12,300, 12,400, 12,500, 12,600, 12,700, 12,800, 12,900, 13,000, 13,100, 13,200, 13,300, 13,400, 13,500, 13,600, 13,700, 13,800, 13,900, or 14,000 cosmids, including all ranges and subranges therebetween. In some embodiments, each of the resulting sequencing silos comprises between 6,000-10,000 cosmids.

In some embodiments, each sequencing silo comprises DNA totaling a length between 105 and 560 million bases (Mb). In some embodiments, each sequencing silo comprises DNA totaling a length of 100 Mb, 101 Mb, 102 Mb, 103 Mb, 104 Mb, 105 Mb, 106 Mb, 107 Mb, 108 Mb, 109 Mb, 110 Mb, 111 Mb, 112 Mb, 113 Mb, 114 Mb, 115 Mb, 116 Mb, 117 Mb, 118 Mb, 119 Mb, 120 Mb, 121 Mb, 122 Mb, 123 Mb, 124 Mb, 125 Mb, 126 Mb, 127 Mb, 128 Mb, 129 Mb, 130 Mb, 131 Mb, 132 Mb, 133 Mb, 134 Mb, 135 Mb, 136 Mb, 137 Mb, 138 Mb, 139 Mb, 140 Mb, 141 Mb, 142 Mb, 143 Mb, 144 Mb, 145 Mb, 146 Mb, 147 Mb, 148 Mb, 149 Mb, 150 Mb, 151 Mb, 152 Mb, 153 Mb, 154 Mb, 155 Mb, 156 Mb, 157 Mb, 158 Mb, 159 Mb, 160 Mb, 161 Mb, 162 Mb, 163 Mb, 164 Mb, 165 Mb, 166 Mb, 167 Mb, 168 Mb, 169 Mb, 170 Mb, 171 Mb, 172 Mb, 173 Mb, 174 Mb, 175 Mb, 176 Mb, 177 Mb, 178 Mb, 179 Mb, 180 Mb, 181 Mb, 182 Mb, 183 Mb, 184 Mb, 185 Mb, 186 Mb, 187 Mb, 188 Mb, 189 Mb, 190 Mb, 191 Mb, 192 Mb, 193 Mb, 194 Mb, 195 Mb, 196 Mb, 197 Mb, 198 Mb, 199 Mb, 200 Mb, 201 Mb, 202 Mb, 203 Mb, 204 Mb, 205 Mb, 206 Mb, 207 Mb, 208 Mb, 209 Mb, 210 Mb, 211 Mb, 212 Mb, 213 Mb, 214 Mb, 215 Mb, 216 Mb, 217 Mb, 218 Mb, 219 Mb, 220 Mb, 221 Mb, 222 Mb, 223 Mb, 224 Mb, 225 Mb, 226 Mb, 227 Mb, 228 Mb, 229 Mb, 230 Mb, 231 Mb, 232 Mb, 233 Mb, 234 Mb, 235 Mb, 236 Mb, 237 Mb, 238 Mb, 239 Mb, 240 Mb, 241 Mb, 242 Mb, 243 Mb, 244 Mb, 245 Mb, 246 Mb, 247 Mb, 248 Mb, 249 Mb, 250 Mb, 251 Mb, 252 Mb, 253 Mb, 254 Mb, 255 Mb, 256 Mb, 257 Mb, 258 Mb, 259 Mb, 260 Mb, 261 Mb, 262 Mb, 263 Mb, 264 Mb, 265 Mb, 266 Mb, 267 Mb, 268 Mb, 269 Mb, 270 Mb, 271 Mb, 272 Mb, 273 Mb, 274 Mb, 275 Mb, 276 Mb, 277 Mb, 278 Mb, 279 Mb, 280 Mb, 281 Mb, 282 Mb, 283 Mb, 284 Mb, 285 Mb, 286 Mb, 287 Mb, 288 Mb, 289 Mb, 290 Mb, 291 Mb, 292 Mb, 293 Mb, 294 Mb, 295 Mb, 296 Mb, 297 Mb, 298 Mb, 299 Mb, 300 Mb, 301 Mb, 302 Mb, 303 Mb, 304 Mb, 305 Mb, 306 Mb, 307 Mb, 308 Mb, 309 Mb, 310 Mb, 311 Mb, 312 Mb, 313 Mb, 314 Mb, 315 Mb, 316 Mb, 317 Mb, 318 Mb, 319 Mb, 320 Mb, 321 Mb, 322 Mb, 323 Mb, 324 Mb, 325 Mb, 326 Mb, 327 Mb, 328 Mb, 329 Mb, 330 Mb, 331 Mb, 332 Mb, 333 Mb, 334 Mb, 335 Mb, 336 Mb, 337 Mb, 338 Mb, 339 Mb, 340 Mb, 341 Mb, 342 Mb, 343 Mb, 344 Mb, 345 Mb, 346 Mb, 347 Mb, 348 Mb, 349 Mb, 350 Mb, 351 Mb, 352 Mb, 353 Mb, 354 Mb, 355 Mb, 356 Mb, 357 Mb, 358 Mb, 359 Mb, 360 Mb, 361 Mb, 362 Mb, 363 Mb, 364 Mb, 365 Mb, 366 Mb, 367 Mb, 368 Mb, 369 Mb, 370 Mb, 371 Mb, 372 Mb, 373 Mb, 374 Mb, 375 Mb, 376 Mb, 377 Mb, 378 Mb, 379 Mb, 380 Mb, 381 Mb, 382 Mb, 383 Mb, 384 Mb, 385 Mb, 386 Mb, 387 Mb, 388 Mb, 389 Mb, 390 Mb, 391 Mb, 392 Mb, 393 Mb, 394 Mb, 395 Mb, 396 Mb, 397 Mb, 398 Mb, 399 Mb, 400 Mb, 401 Mb, 402 Mb, 403 Mb, 404 Mb, 405 Mb, 406 Mb, 407 Mb, 408 Mb, 409 Mb, 410 Mb, 411 Mb, 412 Mb, 413 Mb, 414 Mb, 415 Mb, 416 Mb, 417 Mb, 418 Mb, 419 Mb, 420 Mb, 421 Mb, 422 Mb, 423 Mb, 424 Mb, 425 Mb, 426 Mb, 427 Mb, 428 Mb, 429 Mb, 430 Mb, 431 Mb, 432 Mb, 433 Mb, 434 Mb, 435 Mb, 436 Mb, 437 Mb, 438 Mb, 439 Mb, 440 Mb, 441 Mb, 442 Mb, 443 Mb, 444 Mb, 445 Mb, 446 Mb, 447 Mb, 448 Mb, 449 Mb, 450 Mb, 451 Mb, 452 Mb, 453 Mb, 454 Mb, 455 Mb, 456 Mb, 457 Mb, 458 Mb, 459 Mb, 460 Mb, 461 Mb, 462 Mb, 463 Mb, 464 Mb, 465 Mb, 466 Mb, 467 Mb, 468 Mb, 469 Mb, 470 Mb, 471 Mb, 472 Mb, 473 Mb, 474 Mb, 475 Mb, 476 Mb, 477 Mb, 478 Mb, 479 Mb, 480 Mb, 481 Mb, 482 Mb, 483 Mb, 484 Mb, 485 Mb, 486 Mb, 487 Mb, 488 Mb, 489 Mb, 490 Mb, 491 Mb, 492 Mb, 493 Mb, 494 Mb, 495 Mb, 496 Mb, 497 Mb, 498 Mb, 499 Mb, or 500 Mb, including all ranges and subranges therebetween.

### Metagenomic Library Creation—Silo Pooling Through Barcoding

Persons having skill in the art will recognize that the physical silo pooling described above can be replicated and/or extended, in various degrees, through the use of barcoding technology. DNA Barcodes, also commonly referred to as tags, indexing sequences, or identifier codes, include specific sequences that are incorporated into a nucleic acid molecule for identification purposes. Barcodes can be used to identify individual nucleic acid molecules or groups of nucleic acid molecules.

In some embodiments, the present disclosure teaches using barcodes to silo pool DNA from metagenomic libraries. For example, the present disclosure contemplates barcoding cosmids from E. coli colonies, either individually, or in groups, prior to sequencing. Thus, in some embodiments, the methods of the present disclosure comprise processing and barcoding individual cosmids for NGS.

In some embodiments, the present disclosure teaches traditional use of barcodes to further reduce the complexity of existing sequencing silos. Thus, in some embodiments, the present disclosure teaches the barcoding of individual cosmids.

Certain barcoding embodiments of the present disclosure differ from traditional barcode use, in that the barcodes are not applied to every cosmid, but are instead added to processed sequences in sequencing silos (as described above), or in processed sequences in mini-silo pools, which can then be further pooled into sequencing silos.

In some embodiments, the present disclosure teaches creating mini-silo pools in which a plurality of cosmids are pooled and processed for NGS. In some embodiments each mini silo comprises 100, 200, 300, 400, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000, 30,500, 31,000, 31,500, 32,000, 32,500, 33,000, 33,500, 34,000, 34,500, 35,000, cosmids, including any ranges and subranges therebetween.

In some embodiments, the barcodes are added to mini-silo pools after the pooling has occurred, and after the sequences within each silo have been fragmented into fragment sizes for next generation sequencing. Barcoded mini silo pools could then be further combined into broader sequencing pools before running through the sequencer.

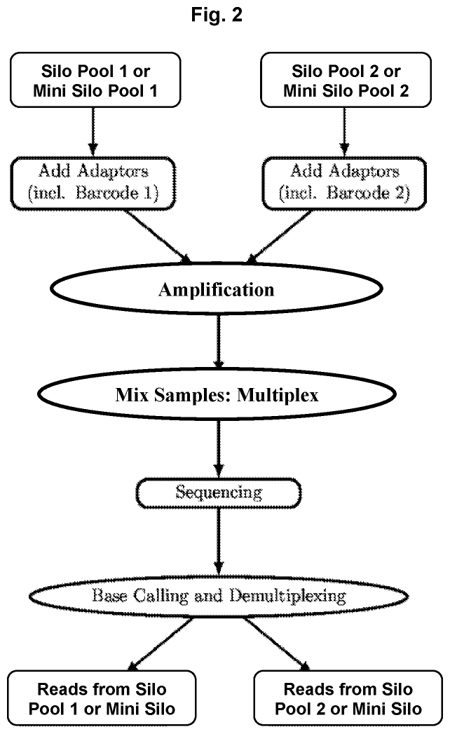

In some embodiments, the individually barcoded sequences are sequenced together with other barcoded samples. The barcoded reads can then be sorted (e.g., de-multiplexed) via known techniques, and assigned to their corresponding groups. (See e.g., FIG. 2).

In other embodiments, the present disclosure teaches that the silo pools described above are processed and barcoded together, and combined with other barcoded silo pools for more efficient use of the sequencer.

Barcodes can be generated based on selecting a particular nucleic acid sequence. For example, the Illumina™ sequencing can utilize 6 bases to effectively generate 48 different barcodes. The Ion Torrent sequencer (e.g., the Ion Proton™ Sequencer or the Ion PGM™ sequencer) can utilize 6 bases to generate 16 barcodes. In some embodiments, rules may be applied to the generation of bar codes that allow for separate barcodes to be correctly identified even if two errors occur during sequencing. Barcoding is described, e.g., in U.S. Pat. No. 7,902,122 and U.S. Pat. Publn. 2009/0098555. Barcode incorporation by primer extension, for example via PCR may be performed using methods described in U.S. Pat. No. 5,935,793 or US 2010/0227329. In some embodiments, a barcode may be incorporated into a nucleic acid via using ligation, which can then be followed by amplification; for example, methods described in U.S. Pat. Nos. 6,261,782, U.S. Pat. Publn. 2011/0319290, or U.S. Pat. Publn. 2012/0028814 may be used with the present invention. In some embodiments, one or more bar code may be used, e.g., as described in U.S. Pat. Publn. 2007/0020640, U.S. Pat. Publn. 2009/0068645, U.S. Pat. Publn. 2010/0273219, U.S. Pat. Publn. 2011/0015096, or U.S. Pat. Publn. 2011/0257031.

Persons having skill in the art will recognize that the nucleic acid sequencing of silo pools, as described above can be replicated and/or potentially improved through the use of synthetic long read technology. In some embodiments, the methods of the present disclosure can be combined with “chromatin capture” technology such as that disclosed in US 2018/0119203, US 2019/0241933, U.S. Pat. Nos. 9,715,573, 10,457,934, and 10,526,641, which are hereby incorporated by reference for all purposes. In some embodiments, barcoding and/or chromatic capture of samples can be automated via commercially-available robotics (e.g., liquid handlers, such as a Tecan) known to persons having skill in the art, or otherwise described in this document. Regardless of the exact implementation of barcodes, the resulting digital assembled libraries should still meet the limitations of digital libraries discussed above. In some embodiments, digital environmental or metagenomic libraries created with barcodes should exhibit N50s of at least 10 kb, 11 kb, 12 kb, 13 kb, 14 kb, or 15 kb. Metagenomic Library Creation—Arraying Library

In some embodiments, the present disclosure teaches methods of creating physical (DNA stock) copies of the multi-gene cluster features set digital metagenomics library. In some embodiments, the physical library copy provides a biological backup copy of digitally stored assembled sequences. In some embodiments, the physical library can be used to conduct further sequencing of one or more silo pool or barcode groups to enhance the sequenced library (e.g., by increasing sequence coverage for one or more portions of the database).

In some embodiments, the physical library provides a mechanism for cloning and studying GIs that are identified through the systems and methods of the present disclosure. That is, in some embodiments, each sequence within the multi-gene cluster features set digital metagenomics library is associated with a location within the physical library, where the relevant DNA can be accessed.

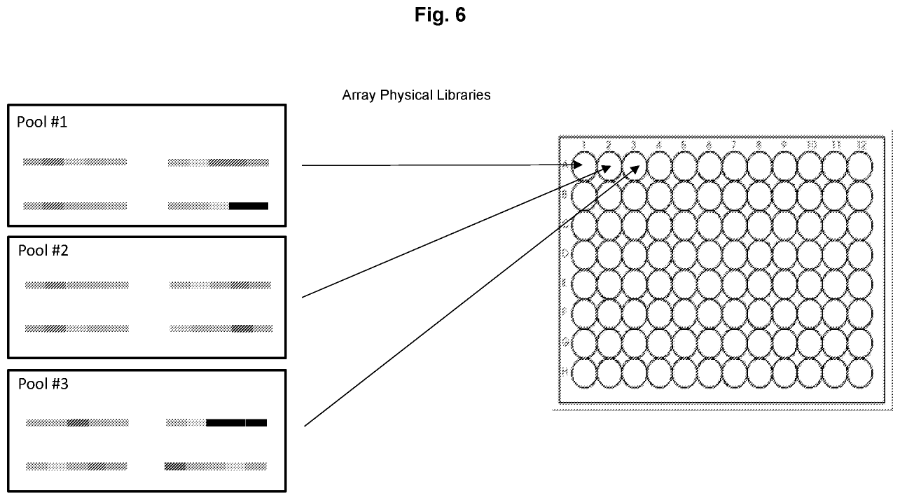

Thus, in some embodiments, cosmid silo pools generated by the methods above are stored in glycerol stocks of E. coli comprising the cosmids. In some embodiments, cosmid silo pools generated by the methods above are stored as isolated DNA stocks. In some embodiments, the physical libraries are stored in 96-well format for easier storage and access. (See step 1 of FIG. 1, and FIG. 6). These physical libraries are herein referred to as “metagenomic physical libraries.”Methods of Producing Digital Metagenomics Libraries—Library Prep and Sequencing

In some embodiments, the resulting silo pools (or cosmids or mini-silo pools) generated above are individually prepared for sequencing. Numerous kits for making sequencing libraries from DNA are available commercially from a variety of vendors. Kits are available for making libraries from microgram down to picogram quantities of starting material. Higher quantities of starting material however require less amplification and can thus better library complexity.

With the exception of Illumina's Nextera prep, library preparation generally entails: (i) fragmentation, (ii) end-repair, (iii) phosphorylation of the 5′ prime ends, (iv) A-tailing of the 3′ ends to facilitate ligation to sequencing adapters, (v) ligation of adapters, and (vi) optionally, some number of PCR cycles to enrich for product that has adapters ligated to both ends. The primary differences in an Ion Torrent workflow are the use of blunt-end ligation to different adapter sequences.

To facilitate multiplexing, different barcoded adapters can be used with each sample. Alternatively, barcodes can be introduced at the PCR amplification step by using different barcoded PCR primers to amplify different samples. High quality reagents with barcoded adapters and PCR primers are readily available in kits from many vendors. However, all the components of DNA library construction are now well documented, from adapters to enzymes, and can readily be assembled into “home-brew” library preparation kits.

An alternative method is the Nextera DNA Sample Prep Kit (Illumina), which prepares genomic DNA libraries by using a transposase enzyme to simultaneously fragment and tag DNA in a single-tube reaction termed “tagmentation.” The engineered enzyme has dual activity; it fragments the DNA and simultaneously adds specific adapters to both ends of the fragments. These adapter sequences are used to amplify the insert DNA by PCR. The PCR reaction also adds index (barcode) sequences. The preparation procedure improves on traditional protocols by combining DNA fragmentation, end-repair, and adaptor-ligation into a single step. This protocol is very sensitive to the amount of DNA input compared with mechanical fragmentation methods. In order to obtain transposition events separated by the appropriate distances, the ratio of transposase complexes to sample DNA can be important. Because the fragment size is also dependent on the reaction efficiency, all reaction parameters, such as temperatures and reaction time, should be tightly controlled for optimal results.

A number of DNA sequencing techniques are known in the art, including fluorescence-based sequencing methodologies (See, e.g., Birren et al., Genome Analysis Analyzing DNA, 1, Cold Spring Harbor, N.Y.). In some embodiments, automated sequencing techniques understood in that art are utilized. In some embodiments, parallel sequencing of partitioned amplicons can be utilized (PCT Publication No WO2006084132). In some embodiments, DNA sequencing is achieved by parallel oligonucleotide extension (See, e.g., U.S. Pat. Nos. 5,750,341; 6,306,597). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803), the 454 picotiter pyrosequencing technology (Margulies et al., 2005 Nature 437, 376-380; US 20050130173), the Solexa single base addition technology (Bennett et al., 2005, Pharmacogenomics, 6, 373-382; U.S. Pat. Nos. 6,787,308; 6,833,246), the Lynx massively parallel signature sequencing technology (Brenner et al. (2000). Nat. Biotechnol. 18:630-634; U.S. Pat. Nos. 5,695,934; 5,714,330), and the Adessi PCR colony technology (Adessi et al. (2000). Nucleic Acid Res. 28, E87; WO 00018957).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al, Nature Rev. Microbiol, 7-287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos Biosciences, and emerging platforms commercialized by VisiGen, Oxford Nanopore Technologies Ltd., Life Technologies/Ion Torrent, and Pacific Biosciences, respectively.

In pyrosequencing (U.S. Pat. Nos. 6,210,891; 6,258,568), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 106 sequence reads can be achieved, resulting in up to 500 million base pairs (Mb) of sequence.

In the Solexa/Illumina platform (Voelkerding et al, Clinical Chem., 55-641-658, 2009; MacLean et al, Nature Rev. Microbiol, 7' 287-296; U.S. Pat. Nos. 6,833,246; 7,115,400; 6,969,488), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55-641-658, 2009; U.S. Pat. Nos. 5,912,148; 6,130,073) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run. In certain embodiments, nanopore sequencing is employed (see, e.g., Astier et al., J. Am. Chem. Soc. 2006 Feb. 8; 128(5):1705-10). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

In some embodiments, the present disclosure teaches use of long-assembly sequencing technology. For example, in some embodiments, the present disclosure teaches PacBio sequencing and/or Nanopore sequencing.

PacBio SMRT technology is based on special flow cells harboring individual picolitre-sized wells with transparent bottoms. Each of the wells, referred to as zero mode waveguides (ZMVV), contain a single fixed polymerase at the bottom (Ardui, S., Race, V., de Ravel, T., Van Esch, H., Devriendt, K., Matthijs, G., et al. (2018b). Detecting AGG interruptions in females with a FMR1 premutation by long-read single-molecule sequencing: a 1 year clinical experience. Front. Genet. 9:150). This allows a single DNA molecule, which is circularized in the library preparation (i.e., the SMRTbell), to progress through the well as the polymerase incorporates labeled bases onto the template DNA. Incorporation of bases induces fluorescence that can be recorded in real-time through the transparent bottoms of the ZMVV (Pollard, M. O., Gurdasani, D., Mentzer, A. J., Porter, T., and Sandhu, M. S. (2018). Long reads: their purpose and place. Hum. Mol. Genet. 27, R234—R241. The average read length for SMRT was initially only ˜1.5 Kb, and with reported high error rate of ˜13% characterized by false insertions (arneiro, M. O., Russ, C., Ross, M. G., Gabriel, S. B., Nusbaum, C., and DePristo, M. A. (2012). Pacific biosciences sequencing technology for genotyping and variation discovery in human data. BMC Genomics 13:375.; Quail, M. A., Smith, M., Coupland, P., Otto, T. D., Harris, S. R., Connor, T. R., et al. (2012). A tale of three next generation sequencing platforms: comparison of Ion Torrent, Pacific Biosciences and Illumina MiSeq sequencers. BMC Genomics 13:341.). Since its introduction, the read length and throughput of SMRT technology have substantially increased. Throughput can reach >10 Gb per SMRT cell for the Sequel machine, while the average read length for both RSII and Sequel is >10 kb with some reads spanning >100 kb (van Dijk, E. L., Jaszczyszyn, Y., Naquin, D., and Thermes, C. (2018). The third revolution in sequencing technology. Trends Genet. 34, 666-681.).

Nanopore sequencing by ONT was introduced in 2015 with a portable MinION sequencer, which was followed by more high-throughput desktop sequencers GridION and PromethlON. The basic principle of nanopore sequencing is to pass a single strand of DNA molecule through a nanopore which is inserted into a membrane, with an attached enzyme, serving as a biosensor (Deamer, D., Akeson, M., and Branton, D. (2016). Three decades of nanopore sequencing. Nat. Biotechnol. 34, 518-524). Changes in electrical signal across the membrane are measured and amplified in order to determine the bases passing through the pore in real-time. The nanopore-linked enzyme, which can be either a polymerase or helicase, is bound tightly to the polynucleotide controlling its motion through the pore (Pollard, M. O., Gurdasani, D., Mentzer, A. J., Porter, T., and Sandhu, M. S. (2018). Long reads: their purpose and place. Hum. Mol. Genet. 27, R234-R241). For nanopore sequencing, there is no clear-cut limitation for read length, except the size of the analyzed DNA fragments. On average, ONT single molecule reads are >10 kb in length but can reach ultra-long for some individual reads lengths of >1 Mb surpassing SMRT (Jain, M., Koren, S., Miga, K. H., Quick, J., Rand, A. C., Sasani, T. A., et al. (2018). Nanopore sequencing and assembly of a human genome with ultra-long reads. Nat. Biotechnol. 36, 338-345). Also, the throughput per run of ONT GridION and PromethlON sequencers are higher than for PacBio (up to 100 Gb and 6 Tb per run, respectively) (van Dijk, E. L., Jaszczyszyn, Y., Naquin, D., and Thermes, C. (2018). The third revolution in sequencing technology. Trends Genet. 34, 666-681).

The present disclosure also teaches use of a technique selected from the group consisting of Hi-C, 3C, 4C, 5C, TLA, TCC, and in situ Hi-C. For example, DNA sequence reads incubating DNA a fixation agent for a period of time to allow crosslinking of the genomic DNA in situ and thereby forming crosslinked genomic DNA; fragmenting the crosslinked genomic DNA; ligating the crosslinked and fragmented genomic DNA to form a proximally ligated complex; shearing the proximally ligated complex to form proximally-ligated DNA fragments; and obtaining a plurality of the proximally-ligated DNA fragments to form a library thereby obtaining the plurality of genomic DNA fragments. For more information on synthetic long reads, see Amarasinghe, S.L., Su, S., Dong, X. et al. Opportunities and challenges in long-read sequencing data analysis. Genome Biol 21, 30 (2020),In some embodiments, the present disclosure teaches hybrid approaches to sequencing the metagenomic library. That is, in some embodiments, the present disclosure teaches sequencing with two or more sequencing technologies (e.g., one short read and one long read). In some embodiments, access to long read sequencing can improve subsequent assembly of the library by providing a reference sequence for DNA regions where the assembly would not otherwise proceed with just the short reads.

### Methods of Producing Digital Metagenomics Libraries—Post-Sequencing Processing and Sequential Assembly

In some embodiments, the present disclosure teaches a sequential sequence assembly method to produce long-assembly sequenced metagenomic libraries, also referred to herein as long DNA contig length metagenomics libraries. Sequence assembly describes the process of piecing together the various sequence reads obtained from the sequencing machine into longer reads representing the original DNA molecule. Assembly is particularly relevant for short-read NGS platforms, where sequences range in the 50-500 base range.

In some embodiments, sequences obtained from the sequencing step can be directly assembled. In some embodiments, the sequences from the sequencing step undergo some processing according to the sequencing manufacturer's instructions, or according to methods known in the art. For example, in some embodiments, the reads from pooled samples are trimmed to remove any adaptor/barcode sequences and quality filtered. In some embodiments, sequences from some sequencers (e.g., Illumina®) are processed to merge paired end reads. In some embodiments, contaminating sequences (e.g. cloning vector, host genome) are also removed. In some embodiments, the methods of the present disclosure are compatible with any applicable post-NGS sequence processing tool. In some embodiments, the sequences of the present disclosure are processed via BBTools (BBMap—Bushnell B.—sourceforge.net/projects/bbmap/).

Sequence assembly techniques can be widely divided into two categories: comparative assembly and de novo assembly. Persons having skill in the art will be familiar with the fundamentals of genome assemblers, which include the overlap-layout-consensus, alignment-layout-consensus, the greedy approach, graph-based schemes and the Eulerian path (Bilal Wajid, Erchin Serpedin, Review of General Algorithmic Features for Genome Assemblers for Next Generation Sequencers, Genomics, Proteomics & Bioinformatics, Volume 10, Issue 2, 2012, Pages 58-73).

According to some embodiments, the assembly of metagenomic library sequences may be a de novo assembly that is assembled using any suitable sequence assembler known in the art including, but not limited to, ABySS, ALLPATHS-LG, AMOS, Arapan-M, Arapan-S, Celera WGA Assembler/CABOG, CLC Genomics Workbench & CLC Assembly Cell, Cortex, DNA Baser, DNA Dragon, DNAnexus, Edena, Euler, Euler-sr, Forge, Geneious, Graph Constructor, IDBA, IDBA-UD, LIGR Assembler, MaSuRCA, MIRA, NextGENe, Newbler, PADENA, PASHA, Phrap, TIGR Assembler, Ray, Sequecher, SeqMan NGen, SGA, SGARCGS, SOPRA, SparseAssembler, SSAKE, SOAPdenovo, SPAdes, Staden gap4 package, Taipan, VCAKE, Phusion assembler, QSRA, and Velvet.

A non-limiting list of sequence assemblers available to date is provided in Table 1.

TABLE 1
Non-limiting List of de novo Sequence Assemblers

| | | Technologies | |
|---|---|---|---|
| Name | Type | and algorithm | Reference/Link |
| ABySS | (large) | Solexa, SOLiD | ABySS 2.0: resource-efficient assembly of large |
| | genomes | De Bruijn | genomes using a Bloom filter. Jackman S D, |
| | | graph(DBG) | Vandervalk B P, Mohamadi H, Chu J, Yeo S, |
| | | | Hammond S A, Jahesh G, Khan H, Coombe L, |
| | | | Warren R L, Birol I. Genome Research, 2017 27: |
| | | | 768-777 |
| ALLPATHS- | (large) | Solexa, | Gnerre S et al. 2010. High-quality draft |
| LG | genomes | SOLiD (DBG) | assemblies of mammalian genomes from |
| | | | massively parallel sequence data. Proceedings of |
| | | | the National Academy of Sciences December 2010, |
| | | | 201017351 |
| AMOS | genomes | Sanger, 454 | //sourceforge.net/projects/amos/ |
| Arapan-M | Medium | All | Sahli and Shibuya. An algorithm for classifying |
| | Genomes | | DNA reads. 2012 International conference on |
| | (e.g. E. coli) | | Bioscience, Biochemistry and Bioinformatics. |
| | | | IPCBEE vol. 31(2012) |
| Arapan-S | Small | All | Sahli M, Shibuya T. Arapan-S: a fast and highly |
| | Genomes | | accurate whole-genome assembly software for |
| | (Viruses and | | viruses and small genomes. BMC Res Notes. |
| | Bacteria) | | 2012; 5: 243. Published 2012 May 16. |
| Celera WGA | (large) | Sanger, 454, | Koren S, Miller J R, Walenz B P, Sutton G. An |
| Assembler/ | genomes | Solexa | algorithm for automated closure during |
| CABOG | | overlap-layout- | assembly. BMC Bioinformatics. 2010; 11: 457. |
| | | consensus(OLC) | Published 2010 Sep. 10. |
| CLC Genomics | genomes | Sanger, 454, | Wingfield B D, Ambler J M, Coetzee M P, et al. |
| Workbench & | | Solexa, SOLiD | IMA Genome-F 6: Draft genome sequences of |
| CLC Assembly | | OLC | Armillaria fuscipes, Ceratocystiopsis minuta, |
| Cell | | | Ceratocystis adiposa, Endoconidiophora |
| | | | laricicola, E. polonica and Penicillium freii |
| | | | DAOMC 242723. IMA Fungus. 2016; 7(1): 217-227. |
| | | | //digitalinsights.qiagen.com |
| Cortex | genomes | Solexa, SOLiD | Whole Genome Sequencing for High-Resolution |
| | | | Investigation of Methicillin Resistant |
| | | | Staphylococcus aureus Epidemiology and |
| | | | Genome Plasticity SenGupta D J, Cummings L, |
| | | | Hoogestraat D R, Butler-Wu S M, Shendure J, |
| | | | Cookson B T, Salipante S J JCM |
| | | | doi: 10.1128/JCM.00759-14 |
| DNA Baser | genomes | Sanger, 454 | www.DnaBaser.com |
| DNA Dragon | genomes | Illumina, | Yoruk, E, Sefer, Ö. (2018). FcMgv1, FcStuA |
| | | SOLiD, | AND FcVeA based genetic characterization in |
| | | Complete | Fusarium culmorum (W. G. Smith). Trakya |
| | | Genomics, 454, | University Journal of Natural Sciences, 19 (1), 63-69. |
| | | Sanger | www.dna-dragon.com/ |
| Edena | genomes | Illumina | Analysis of the salivary microbiome using |
| | | OLC | culture-independent techniques. Lazarevic V, |
| | | | Whiteson K, Gaia N, Gizard Y, Hernandez D, |
| | | | Farinelli L, Osteras M, Francois P, Schrenzel J. |
| | | | J Clin Bioinforma. 2012 Feb. 2; 2: 4. |
| Euler-sr | genomes | 454, Solexa | Chaisson and Pevzner. Short read fragment |
| | | | assembly of bacterial genomes. Genome |
| | | | Res. 2008. 18: 324-330 |
| Forge | (large) | 454, Solexa, | DiGuistini, S., Liao, N. Y., Platt, D. et al. De |
| | genomes, | SOLiD, Sanger | novo genome sequence assembly of a filamentous |
| | EST, | | fungus using Sanger, 454 and Illumina sequence |
| | metagenomes | | data. Genome Biol 10, R94 (2009). |
| | | | https://doi.org/10.1186/gb-2009-10-9-r94 |
| Geneious | genomes | Sanger, 454, | www.geneious.com/features/assembly-mapping/ |
| | | Solexa, Ion | |
| | | Torrent, | |
| | | Complete | |
| | | Genomics, | |
| | | PacBio, Oxford | |
| | | Nanopore, | |
| | | Illumina | |
| IDBA (Iterative | (large) | Sanger, 454, Solexa | Peng, Y., et al. (2010) IDBA- A Practical Iterative |
| De Bruijn | genomes | | de Bruijn Graph De Novo Assembler. RECOMB. |
| graph short | | | Lisbon. |
| read | | | |
| Assembler) | | | |
| MaSuRCA | (large) | Sanger, Illumina, | Zimin, A. et al. The MaSuRCA genome |
| (Maryland | genomes | 454 | Assembler. Bioinformatics (2013). |
| Super Read - | | hybrid approach | doi: 10.1093/bioinformatics/btt476 |
| Celera | | | |
| Assembler) | | | |
| MIRA | genomes, | Sanger, 454, | Chevreux et al. (2004) Using the miraEST |
| (Mimicking | ESTs | Solexa | Assembler for Reliable and Automated mRNA |
| Intelligent Read | | | Transcript Assembly and SNP Detection in |
| Assembly) | | | Sequenced ESTs Genome Research 2004. |
| | | | 14: 1147-1159. |
| NextGENe | (small | 454, Solexa, | Manion et al. De novo assembly of short sequence |
| | genomes?) | SOLiD | reads with nextgene ™ software & condensation |
| | | | tool. Application |
| | | | note//softgenetics.com/PDF/DenovoAssembly_SSR_AppNote.pdf |
| Newbler | genomes, | 454, | Margulies M et al. Genome sequencing in |
| | ESTs | Sanger(OLC) | microfabricated high-density picolitre |
| | | | reactors. Nature. 2005 Sep. 15; 437(7057): 376-80. |
| PADENA | genomes | 454, Sanger | Thareja, G.; Kumar, V.; Zyskowski, M.; Mercer, |
| | | | S. and Davidson, B. (2011). PadeNA: A |
| | | | PARALLEL DE NOVO |
| | | | ASSEMBLER. In Proceedings of the |
| | | | International Conference on Bioinformatics |
| | | | Models, Methods and Algorithms - Volume 1: |
| | | | BIOINFORMATICS, (BIOSTEC 2011) |
| PASHA | (large) | Illumina | Liu, Y., Schmidt, B. & Maskell, D. L. Parallelized |
| | genomes | | short read assembly of large genomes using de |
| | | | Bruijn graphs. BMC Bioinformatics 12, 354 |
| | | | (2011) |
| Phrap | genomes | Sanger, 454, | Bastide and Mccombie, Assembling Genomic |
| | | Solexa | DNA sequences with PHRAP. Current protocols |
| | | (OLC) | in Bioinformatics. Vol 17(1) March 2007. |
| TIGR Assembler | genomic | Sanger | Sutton G G, White O, Adams M D, Kerlavage A R |
| | | | (1995) TIGR Assembler: A new tool for |
| | | | assembling large shotgun sequencing projects. |
| | | | Genome Science and Technology 1: 9-19. |
| Ray | genomes | Illumina, mix of | Boisvert et al. Ray Meta: scalable de novo |
| | | Illumina and | metagenome assembly and profiling. Genome |
| | | 454, paired or | Biology (BioMed Central Ltd). 13: R122, |
| | | not | Published: 22 Dec. 2012 |
| Sequencher | genomes | traditional and | Bromberg C. Gene Codes Corporation; 1995. |
| | | next generation | Sequenche |
| | | sequence data | |
| SeqMan NGen | (large) | Illumina, ABI | Feldmeyer B et al. Short read Illumina data for the |
| | genomes, | SOLiD, Roche | de novo assembly of a non-model snail species |
| | exomes, | 454, Ion Torrent, | transcriptome (Radix balthica, Basommatophora, |
| | transcriptomes, | Solexa, Sanger | Pulmonata), and a comparison of assembler |
| | metagenomes, | | performance. BMC Genomics. 2011; 12: 317. |
| | ESTs | | Published 2011 Jun. 16. |
| | | | www.dnastar.com/t-products-seqman-ngen.aspx |
| SGA | (large) | Illumina, Sanger | Simpson J T and Durbin R. Efficient de novo |
| | genomes | (Roche 454?, Ion | assembly of large genomes using compressed data |
| | | Torrent?) | structures. Genome Res. 2012; 22(3): 549-556 |
| SHARCGS | (small) | Solexa | Dohm J C et al., Substantial biases in ultra-short |
| | genomes | | read data sets from high-throughput DNA |
| | | | sequencing |
| | | | Nucleic Acids Res. 2008 Jul. 26. |
| SOPRA | genomes | Illumina, | Dayarian, A. et al., SOPRA: Scaffolding |
| | | SOLiD, Sanger, | algorithm for paired reads via statistical |
| | | 454 | optimization. BMC Bioinformatics 11, 345 (2010) |
| SparseAssembler | (large) | Illumina, 454, | Ye, C., Ma, Z. S., Cannon, C. H. et al. Exploiting |
| | genomes | Ion torrent | sparseness in de novo genome assembly. BMC |
| | | | Bioinformatics 13, S1 (2012). |
| SSAKE | (small) | Solexa (SOLiD? | Warren R L, Sutton G G, Jones S J M, Holt R A. |
| | genomes | Helicos?) | 2007 (epub 2006 Dec. 8). |
| | | | Assembling millions of short DNA sequences |
| | | | using SSAKE. Bioinformatics 23: 500 |
| SOAPdenovo | genomes | Solexa | Luo, Ruibang et al. “SOAPdenovo2: an |
| | | (DBG) | empirically improved memory-efficient short- |
| | | | read de novo assembler.” GigaScience vol. 1, 1 18. |
| | | | 27 Dec. 2012, doi: 10.1186/2047-217X-1-18 |
| SPAdes | (small) | Illumina, Solexa | Bankevich A. et al., SPAdes: A New Genome |
| | genomes, | | Assembly Algorithm and Its Applications to |
| | single-cell | | Single-Cell Sequencing. Journal of |
| | | | Computational Biology, 2012 |
| Staden gap5 | BACs (, small | Sanger | Bonfield, James K. and Whitwham, Andrew. |
| package | genomes?) | | Gap5 - editing the billion fragment sequence |
| | | | assembly. Bioinformatics 26, 1699-1703, (2010) |
| Taipan | (small) | Illumina | Bertil Schmidt et al, A fast hybrid short read |
| | genomes | | fragment assembly algorithm, Bioinformatics, |
| | | | Volume 25, Issue 17, 1 Sep. 2009, Pages |
| | | | 2279-2280 |
| VCAKE | (small) | Solexa (SOLiD?, | William R. Jeck et al., Extending assembly of |
| | genomes | Helicos?) | short DNA sequences to handle |
| | | | error, Bioinformatics, Volume 23, Issue 21, 1 |
| | | | Nov. 2007, Pages 2942-2944, |
| Phusion | (large) | Sanger | Mullikin, James C, and Zemin Ning. “The |
| assembler | genomes | (OLC) | phusion assembler.” Genome research vol. 13, 1 |
| | | | (2003): 81-90. doi: 10.1101/gr.731003 |
| Quality Value | genomes | Sanger, Solexa | Bryant, Douglas W Jr et al. “QSRA: a quality- |
| Guided SRA | | | value guided de novo short read assembler.” BMC |
| (QSRA) | | | bioinformatics vol. 10 69. 24 Feb. 2009, |
| | | | doi: 10.1186/1471-2105-10-69 |
| Velvet | (small) | Sanger, 454, | Zerbino, Daniel R. “Using the Velvet de novo |
| | genomes | Solexa, SOLiD | assembler for short-read sequencing |
| | | (DBG) | technologies.” Current protocols in |
| | | | bioinformatics vol. Chapter 11 (2010): Unit 11.5. |
| | | | doi: 10.1002/0471250953.bi1105s31 |

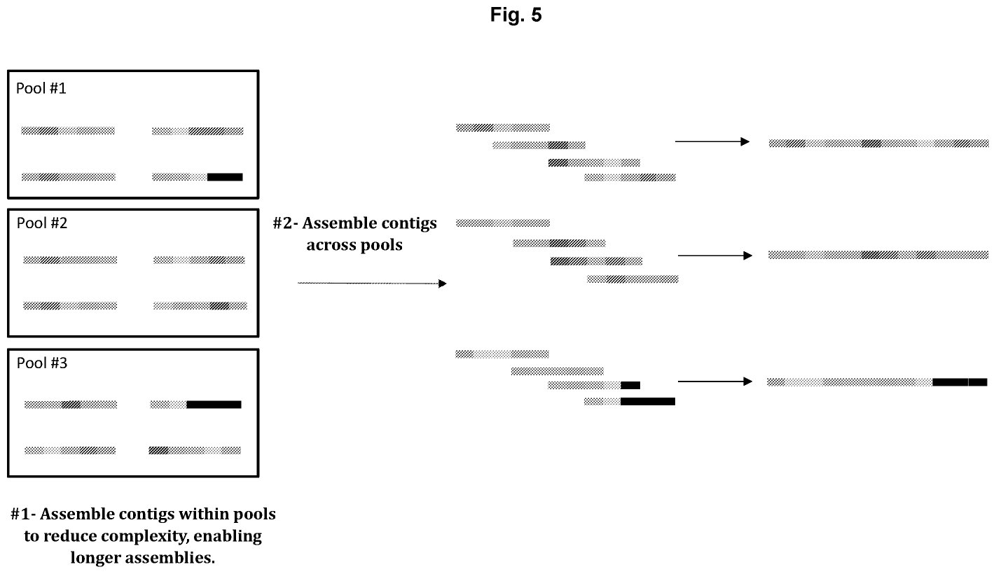

In some embodiments, the present disclosure teaches a sequential assembly technique comprising at least a first assembly and a second assembly. In some embodiments, the first assembly is an assembly of sequences from each silo pool (or if barcoded, to any distinctly barcoded group of sequences). This first assembly thus only builds sequences by combining reads obtained from within the same silo pool (or barcoded group). This first assembly benefits from a relatively lower complexity pool of reads, and is therefore able to align sequences with higher confidence (and thus generate longer assemblies compared to more complex pools). The resulting sequences from the first assembly comprise a plurality of mini metagenomes, each corresponding to a portion of a cosmid in the initial E. coli cosmid library. (See FIG. 5). In some embodiments, the mini-metagenome library comprises DNA of intermediary DNA contig length, wherein the average sequenced and assembled DNA contig length is at least about 10 kb.

In some embodiments, the mini metagenomes from the first assembly have an average sequence length of about 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, or 40 kb , including all ranges and subranges therebetween.

In some embodiments, the resulting assemblies from the first assembly are then used to prepare longer assemblies across different silo pools (or barcoded groups) in a second assembly. The output of the second assembly is a metagenomics library comprising DNA of long contig length. As described above, each of the silo pools (or barcoded groups) used for the first assembly are smaller portions of the starting metagenomic DNA sample. Thus, it is possible, and even likely, that sequences contained in one silo pool/barcode group may correspond (i.e., assemble, align) with sequences from one or more other silo pools/barcode groups. Thus, in some embodiments, each of the assembled mini metagenomes from the first assembly are provided as input for a second assembly. In some embodiments, mini metagenomes from the first assembly can be combined, and result in longer sequence assemblies. (See FIG. 5). In some embodiments, the second assembly also comprises assembling any unassembled reads remaining from each of the silo pools/barcode groups.

In some embodiments, the resulting cross-silo/barcode group assemblies produce even larger sequence strings. The resulting assembled sequences from the first and/or second assembly steps are populated into a database and were referred to as a “digital metagenomics library.”

In some embodiments, the resulting digital metagenomics library comprises an average sequence length of about 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb, 50 kb, 51 kb, 52 kb, 53 kb, 54 kb, 55 kb, 56 kb, 57 kb, 58 kb, 59 kb, 60 kb, 61 kb, 62 kb, 63 kb, 64 kb, 65 kb, 66 kb, 67 kb, 68 kb, 69 kb, 70 kb, 71 kb, 72 kb, 73 kb, 74 kb, 75 kb, 76 kb, 77 kb, 78 kb, 79 kb, 80 kb, 81 kb, 82 kb, 83 kb, 84 kb, 85 kb, 86 kb, 87 kb, 88 kb, 89 kb, 90 kb, 91 kb, 92 kb, 93 kb, 94 kb, 95 kb, 96 kb, 97 kb, 98 kb, 99 kb, 100 kb, 101 kb, 102 kb, 103 kb, 104 kb, 105 kb, 106 kb, 107 kb, 108 kb, 109 kb, 110 kb, 111 kb, 112 kb, 113 kb, 114 kb, 115 kb, 116 kb, 117 kb, 118 kb, 119 kb, 120 kb, 121 kb, 122 kb, 123 kb, 124 kb, 125 kb, 126 kb, 127 kb, 128 kb, 129 kb, or 130 kb, including all ranges and subranges therebetween. In some embodiments, the average sequence length of the digital metagenomics library is 32 kb. In some embodiments, the digital metagenomics library comprises a plurality of sequenced and assembled DNA contigs with a length of at least about 10 kb, 15, kb, 20 kb, 30 kb, 35, kb, 40 kb, 45 kb, 50 kb, or 100 kb. In some embodiments, the digital metagenomics library comprises sequenced and assembled DNA contigs having an average length of at least about 10 kb, 15, kb, 20 kb, 30 kb, 35, kb, or 40 kb.

In some embodiments, the digital metagenomics library is at least about 50 MB, 75 MB, 100 MB, 200 MB, 300 MB, 400 MB, or 500 MB in size.

In some embodiments, the digital metagenomics library comprising GI-associated feature sets comprises sequenced and digitally assembled contig sequences having an average length of at least about 10 kb or 20 kb and a library size of at least about 500 MB.

In some embodiments, the digital metagenomics library comprising GI-associated feature sets comprises sequenced and digitally assembled contig sequences having an average length of at least about 10 kb or 20 kb and a library size of at least about 1 TB.

In some embodiments, the digital metagenomics library comprising GI-associated feature sets comprises sequenced and digitally assembled contig sequences having an average length of at least about 10 kb or 20 kb and a library size of between about 500 MB and 1 TB.

### In Silico Discovery of Insecticidal Protein Encoding GIs

### Insecticidal Gene Search Workflow

In some embodiments, the present disclosure teaches in silico methods for searching a digital metagenomics library and identifying an insecticidal protein of interest. In some embodiments, the methods of the present disclosure comprise the steps of : a) querying a digital metagenomics library for a signal indicative of a GI; b) supplying the output of said query as a plurality of GI-associated digital feature sets comprising all gene open reading frames within a predetermined threshold parameter from the signal indicative of the GI; c) screening the GI-associated digital feature sets for additional indications of an insecticidal protein encoding gene; and d) selecting one or more candidate insecticidal protein encoding gene(s) from among the GI-associated digital feature sets screened in step (c), thereby identifying a candidate insecticidal protein from the digital metagenomics library.

Although the present disclosure presents steps (a), (b), and (c) sequentially, persons having skill in the art will understand that, in some embodiments the identifications of steps (a) (b) and (c) can be performed in any order. For example, in some embodiments, all the ORFs of within a digital metagenomic library can be queried for additional indications of an insecticidal protein before that same digital metagenomics library is queried for a signal indicative of a PAL In this embodiment, the selection step (d) would comprise selecting one or more candidate insecticidal protein that included an “additional indication of an insecticidal protein,” as determined first in step (c), and which was also within the predetermined threshold of a “signal indicative of a GI,” as determined second in step (a).

Each of these steps is discussed in more detail below.

### Querying a Digital Metagenomics Library for a Signal Indicative of a GI

In some embodiments the querying step comprises searching the digital metagenomics library for a signal indicative of a GI. In some embodiments, the output of the querying step is a plurality of GI-associated digital feature sets comprising all gene open reading frames within a predetermined threshold parameter from the signal indicative of the GI. As used herein, a “GI-associated digital feature set” comprises DNA digital sequences from the digital metagenomics library that are identified as comprising a candidate GI, and which (optionally) further survive the filtering steps described throughout this disclosure.

In some embodiments, the predetermined threshold parameter is the length of sequence (e.g., measured in bases) contained within a GI-associated digital feature set. In some embodiments, the predetermined threshold parameter is measured from the ends of the sequence comprising a signal indicative of a GI. In some embodiments, the predetermined threshold parameter represents the entire length of the digital feature set (e.g., 10 kb total length, with the signal indicative of a GI in optionally in the center).

In other embodiments, the predetermined threshold parameter is provided as the distance (e.g., in base pairs) upstream and/or downstream from the signal indicative of a GI. Thus, in some embodiments, the predetermined threshold parameter is the number of downstream base pairs from the 3′ end of the signal indicative of a GI and the number of upstream base pairs from the 5′ end of the signal indicative of the GI.

In some embodiments, the 3′ end of a signal indicative of a GI is measured from the end of the open reading frame (stop codon) of a signal indicative of a GI. In some embodiments, the 3′ end of a signal indicative of a GI is measured from the end of the 3′ untranslated region (3′ UTR) of a signal indicative of a GI. In some embodiments, the 3′ end of a signal indicative of a GI is measured from the last nucleotide within the signal indicative of the GI.

In some embodiments, the 5′ end of a signal indicative of a GI is measured from the beginning of the open reading frame (start codon) of a signal indicative of a GI. In some embodiments, the 5′ end of a signal indicative of a GI is measured from the beginning of the 5′ untranslated region (5′ UTR) of a signal indicative of a GI. In some embodiments, the 5′ end of a signal indicative of a GI is measured from the first nucleotide within the signal indicative of the GI.

In some embodiments, there is more than one signal indicative of a GI within a GI-associated digital feature set. In some embodiments, different GI-associated digital feature sets are produced for each signal indicative of a GI. For example, the presence of three signals associated with a GI within a genomic region may yield three separate (but perhaps overlapping) GI-associated digital feature sets.

In some embodiments, the GI-associated digital feature sets are expanded to include all signals indicative of a GI that are located within the GI-associated digital feature set, and the predetermined threshold parameter is calculated from the ends of the outermost signals indicative of a GI. For example, in some embodiments, the GI-associated digital feature set is produced based on a single signal indicative of a GI, as described above. If the digital feature set encompasses other signals indicative of the GI, then the digital feature set is produced again, this time measuring the predetermined threshold parameter from the outermost signal indicative of a GI. Thus, in some embodiments, the GI digital feature set comprising three signals indicative of a GI includes the sequence within the predetermined threshold parameter as measured from the beginning of the most upstream signal indicative of a GI, and the sequence within predetermined threshold parameter measured from the end of the most downstream signal indicative of a GI.

In some embodiments, the predetermined threshold parameter is less than about 5 kb, 6 kb, 7 kb, 8 kb, 9 kb, 10 kb, 15 kb, 20 kb, 25 kb, 30 kb, 35 kb, 40 kb, 45 kb, 50 kb, 55 kb, 60 kb, kb, 70 kb, 75 kb, 80 kb, 85 kb, 90 kb, 95 kb, or 100 kb from the 5′ end of the signal indicative of the GI. In some embodiments, the predetermined threshold parameter is less than about 5 kb, 6 kb, 7 kb, 8 kb, 9 kb, 10 kb from the signal indicative of the GI.

In some embodiments, the predetermined threshold parameter is less than about 5 kb, 6 kb, 7 kb, 8 kb, 9 kb, 10 kb, 15 kb, 20 kb, 25 kb, 30 kb, 35 kb, 40 kb, 45 kb, 50 kb, 55 kb, 60 kb, kb, 70 kb, 75 kb, 80 kb, 85 kb, 90 kb, 95 kb, or 100 kb from the 3′ end of the signal indicative of the GI. In some embodiments, the predetermined threshold parameter is less than about 5 kb, 6 kb, 7 kb, 8 kb, 9 kb, 10 kb from the signal indicative of the GI.

In some embodiments, the predetermined threshold parameter is less than about 5 kb, 6 kb, 7 kb, 8 kb, 9 kb, 10 kb, 15 kb, 20 kb, 25 kb, 30 kb, 35 kb, 40 kb, 45 kb, 50 kb, 55 kb, 60 kb, kb, 70 kb, 75 kb, 80 kb, 85 kb, 90 kb, 95 kb, or 100 kb in total length, with the signal indicative of the GI in the center.

In some embodiments, signals indicative of a GI comprise be one or more of the following: a gene encoding an integrase, a gene encoding a transposase, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein or a homolog thereof (e.g., as identified via sequence identity or HMM as presently disclosed), a gene encoding a virulence factor, a gene encoding a tRNA, a gene containing a domain of unknown function, a prophage gene, or a phage gene. In some embodiments the signal indicative of a GI is identified via one or more of the methods disclosed in the specification (e.g., sequence identity, similarity, HMM, etc, which predict the relevant functionality of a sequence).

This document uses the term “signal indicative of a GI” and “GI indicator” in are interchangeable.”

In some embodiments, the GI-associated digital feature sets comprise 3, 4, 5, 6, 7, 8, 9, or more GI indicators.

In some embodiments, the GI indicator is the presence of a gene encoding an integrase. An integrase is an enzyme that catalyzes horizontal transfer of DNA.

In some embodiments, the GI indicator is the presence of a gene encoding an transposase. The term “transposase” refers to a polypeptide that catalyzes the excision of a transposon from a donor polynucleotide, for example a vector, and (providing the transposase is not integration-deficient) the subsequent integration of the transposon into the genomic or extrachromosomal DNA of a target cell. The transposase binds a transposon end. Transposases have been identified in a wide range of organisms, including Argyrogramma agnate (GU477713), Anopheles gambiae (XP 312615; XP 320414; XP 310729), Aphis gossypii (GU329918), Acyrthosiphon pisum (XP 001948139), Agrotis ipsilon (GU477714), Bombyx mori (BAD 11135), Ciona intestinalis (XP 002123602), Chilo suppressalis (JX294476), Drosophila melanogaster (AAL39784), Daphnia pulicaria (AAM76342), Helicoverpa armigera (ABS18391), Homo sapiens (NP 689808), Heliothis virescens (ABD76335), Macdunnoughia crassisigna (EU287451), Macaca fascicularis (AB 179012), Mus musculus (NP 741958), Pectinophora gossypiella (GU270322), Rattus norvegicus (XP 220453), Tribolium castaneum (XP 001814566), Trichoplusia ni (AAA87375) and Xenopus tropicalis (BAF82026).

In some embodiments, the GI indicator is the presence of a gene encoding an endonuclease. The term endonuclease refers to any wild-type or variant enzyme capable of catalyzing the hydrolysis (cleavage) of a bond between nucleic acids within a DNA or RNA molecule. include type II restriction endonucleases, such as FokI, HhaI, HindIII, NotI, BbvCI, EcoRI, BglII and AlwI. Endonucleases also include rare-cutting endonucleases when typically having a polynucleotide recognition site of about 12-45 base pairs (bp), more preferably 14-45bp in length. Rarely cutting endonucleases induce DNA Double Strand Breaks (DSBs) at defined loci.

In some embodiments, the GI indicator is the presence of a gene encoding an adhesin. The term “adhesin” as used herein includes proteins and peptides which can bind to extracellular matrix proteins and/or mediate adherence to host cells. In some embodiments, the adhesin is FimH. In some embodiments, the adhesin belongs to the Dr family of adhesins. The Dr family of adhesins bind to the Dr blood group antigen component of decay-accelerating factor (DAF). In some embodiments, the adhesin belongs to the multivalent adhesion molecules (MAM) family. MAMs contain tandem repeats of mammalian cell entry (MCE) domains which specifically bind to extracellular matrix proteins and anionic lipids on host tissues. In some embodiments, the adhesin is K88 or CFA1 of Escherichia coli. In some embodiments the adhesin belongs to the trimeric autotransporter adhesins (TAAs) family of adhesins. TAAs are proteins found on the outer membrane of Gram-negative bacteria.

In some embodiments, the GI indicator is the presence of a gene encoding a secretion system. The term “secretion system” refers to proteins or protein complexes on the cell membranes of microbes responsible for secretion of substances. In some embodiments, the secretion system secretes unfolded proteins. In some embodiments, the secreted protein is sent to the inner membrane or the periplasm. In some embodiments, the secretion system secretes folded proteins. In some embodiments, the secretion system secretes ions, carbohydrates, drugs, or proteins. In some embodiments, the secretion system is selected from the group consisting of the SecA, signal recognition particle pathway (SRP), twin arginine translocation (Tat), type 1 secretion system, type 2 secretion system, secretin, type 3 secretion system, type 4 secretion system, type 5 secretion system, type 6 secretion system, type 7 secretion system, and type 9 secretion system.

In some embodiments, the GI indicator is the presence of a gene encoding a toxin (e.g. having sequence similarity to known toxin genes). In some embodiments, the known toxin is selected from the group consisting of a Fit, makes caterpillars floppy (Mcf), a Tc, vegetative insecticidal proteins (Vip), anthrax, botulinum, and crystal (Cry) toxins. In some embodiments, the known toxin is an endotoxin. In some embodiments, the known toxin is an exotoxin.

In some embodiments, the GI indicator is the presence of a gene encoding an invasin. The term “invasin” refers to a protein that allows a microbe to penetrate a host cell. In some embodiments, the invasin is selected from the group consisting of a hyaluronidase, a collagenase, a neuraminidase, a streptokinase, a staphylokinase, a lecithinase, a phospholipase, a leukocidin, a phospholipase, a lecithinase, a hemolysin, and a coagulase. In some embodiments, the invasin is SipB, SipC, SipD, SipA, or SipE (e.g., from Shigella or Salmonella) or a homolog thereof. In some embodiments, the invasin is IpaB, IpaC, or IpaD (e.g., from Shigella spp) or a homolog thereof. In some embodiments, the invasin is YopB, YopD, or LcrV (e.g., from Yersinia spp) or a homolog thereof. In some embodiments, the invasin is PopB, PopD, or PcrV (e.g., from Pseudomonas spp) or a homolog thereof. In some embodiments, the invasin is EspA, EspD, or EspB (e.g., from enteropathogenic Escherichia coli) or a homolog thereof. In some embodiments, the invasin is invasin (inv) from Yersinia pseudotuberculosis or Yersinia enterocolitica, or a homolog thereof.

In some embodiments, the GI indicator is the presence of a gene encoding a modulin. In some embodiments, the modulin is a phenol-soluble modulin. The term “phenol-soluble modulin” refers to a class of amphipathic, α-helical peptides secreted by bacteria in the genus Staphylococcus. Phenol-soluble modulins serve various functions, including serving as toxins, assisting in biofilm formation, and assisting with cell lysis, invasion, and infection. In some embodiments, the phenol-soluble modulin is selected from the group consisting of PSM gamma, PSM delta, PSM alpha, PSM alpha 3, and PSM beta.

In some embodiments, the GI indicator is the presence of a gene encoding an iron uptake system. Iron is a vital element for microorganisms due to its role in oxidation reduction reactions. However, inorganic ion is neither available in aerobic environments nor in biological systems. In some embodiments, a GI comprises an iron uptake system. An iron uptake system enables a microbe to sequester iron, thus defending against invading pathogens. In some embodiments, an iron uptake system comprises one or more of the following: a siderophore (a low-molecular-weight high-affinity iron ligands), an outer membrane (OM) receptor for binding host iron-chelating proteins, a ferrous iron transporters, a transferrin, a lactoferrin, a transport system for heme or hemoproteins, or an ABC transporter. In some embodiments, an iron uptake system comprises an ABC transporter. In some embodiments, an ABC transporter comprises one or more of the following: a ligand-binding protein (in the periplasm in gram-negative bacteria), an inner membrane permease, and an ATP hydrolase.

In some embodiments, the GI indicator is the presence of a gene encoding a protease. The term “protease” refers to a protein that catalyzes the breakdown of proteins into smaller polypeptides or single amino acids. Non-limiting examples of proteases include SigA, trypsin, chymotrypsin, endoproteinase Asp-N, endoproteinase Arg-C, endoproteinase Glu-C, endoproteinase Lys-C, thermolysin, papain, clostripain, cathepsin C, and carboxypeptidase P.

In some embodiments, the GI indicator is the presence of a gene encoding a known insecticidal protein or homolog thereof. In some embodiments, the insecticidal protein is selected from the group consisting of Fit, makes caterpillars floppy (Mcf), vegetative insecticidal proteins (Vip), a toxin complex (Tc), and Cry proteins. In some embodiments, the presence of a known insecticidal protein or homolog thereof increases the likelihood that an additional (e.g., a second, a third, a fourth, a fifth, a sixth, a seventh, an eighth, a ninth, or tenth) insecticidal gene is nearby (e.g., within the predetermined threshold

In some embodiments, the GI indicator is the presence of a gene encoding a virulence factor. The term “virulence factor” refers to molecules produced by microbes that add to their effectiveness and enable them to achieve the following: colonization of a niche in the host, immunoevasion, or evasion of the host's immune response. Non-limiting examples of virulence factors include toxins, surface coats that inhibit phagocytosis, and surface receptors that bind to host cells. Non-limiting examples of the virulence factors include BslA (e.g., from Bacillus), OatA (e.g., from Listeria), YadA (e.g., from Yersinia), AdsA (e.g., from Staphylococcus) or any homolog thereof.

In some embodiments, the GI indicator is the presence of a gene encoding a tRNA. In some embodiments, a tRNA gene serves as an anchor point for insertion into a genome. In some embodiments, the tRNA gene accepts an amino acid selected from the group consisting of histidine, lysine, arginine, glutamine, asparagine, glutamic acid, aspartic acid, proline, glycine, serine, threonine, phenylalanine, alanine, methionine, isoleucine, leucine, tyrosine, valine, tryptophan, cysteine, and selenocysteine.

In some embodiments, the GI indicator is the presence of a gene encoding a domain of unknown function (DUF). Up to 50% of genes in some GIs consist of proteins of unknown function (e.g., P. aeroginosa GIs). The term “domain of unknown function” refers to a protein with no characterized function. The Pfam database contains the sequences of many microbial DUFs. The following article describes bacterial DUFs and is incorporated by reference herein in its entirety: Goodacre et al. mBio Dec 2013, 5 (1) e00744-13.

In some embodiments, the GI indicator is the presence of a gene encoding a prophage or phage. The term phage, also referred to as “bacteriophage,” is a virus that infects and replicates within bacteria and archaea. The term prophage refers to a bacteriophage genome that is integrated into inserted and integrated into a circular bacterial DNA chromosome or exists as an extrachromosomal plasmid. In some embodiments, the sequence of a gene encoding a phage is found in the National Center for Biotechnology Information (NCBI) phage database. In some embodiments, any of the following tools may be used to identify prophage sequences: MARVEL, Prophage Hunter, PHAS IER, PhiSpy, MetaPhinder, or VirFinder. The following article describes tools used to identify prophage sequences and is incorporated by reference herein in its entirety: Song. Nucleic Acids Res. 2019 Jul. 2; 47(W1): W74-W80.

In some embodiments, the GI indicator is a switch in GC content. The term “GC content” as used herein refers to the percentage of nitrogenous bases in a nucleic acid molecule or within a portion of a nucleic acid (e.g., a stretch of DNA of about 100 bases, about 200 bases, about 300 bases, about 400 bases, about 500 bases, about 600 bases, about 700 bases, about 800 bases, about 900 bases, about 1000 bases, about 2 kb, about 3 kb, about 4 kb, about 5 kb, about 6 kb, about 7 kb, about 8 kb, about 9 kb, or about 10 kb of a nucleic acid) that contain either guanosine or cytosine. In some embodiments, the GI indicator is a switch from low GC content to high GC content. In some embodiments, the GI indicator is a switch from high GC content to low GC content. The term “high GC content” refers to a GC content that is greater than or equal to 65%, for example, 65%, about 70%, about 75%, about 80%, about 85%, about 90%, about 95%, or about 100%. The term “low GC content” refers to a GC content that is less than 65%, for example, about 60%, about 55%, about 50%, about 45%, about 40%, about 35%, about 30%, about 25%, about 20%, about 15%, about 10%, or about 5%. In some embodiments, the GC content switches from high GC content to low GC content over about 100 bases, over about 200 bases, over about 300 bases, over about 400 bases, over about 500 bases, over about 600 bases, over about 700 bases, over about 800 bases, over about 900 bases, over about 1000 bases, over about 2 kb, over about 3 kb, over about 4 kb, over about 5 kb, over about 6 kb, over about 7 kb, over about 8 kb, over about 9 kb, or about 10 kb. In some embodiments, the GC content switches from low GC content to high GC content over about 100 bases, over about 200 bases, over about 300 bases, over about 400 bases, over about 500 bases, over about 600 bases, over about 700 bases, over about 800 bases, over about 900 bases, over about 1000 bases, over about 2 kb, over about 3 kb, over about 4 kb, over about 5 kb, over about 6 kb, over about 7 kb, over about 8 kb, over about 9 kb, or about 10 kb. In some embodiments, the switch from low GC content to high GC content is a GC content change of at least 10%, 20%, 30%, 40%, 50%, 60%, 70%, 80%, or 90% over the course of less than about 100 bases, about 200 bases, about 300 bases, about 400 bases, about 500 bases, about 600 bases, about 700 bases, about 800 bases, about 900 bases, about 1000 bases, about 2 kb, about 3 kb, about 4 kb, about 5 kb, about 6 kb

In some embodiments, the signal indicative of a GI is a homolog to a known gene encoding an integrase, a gene encoding a transposase, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage gene, or a phage gene, thereby enabling identification of the GI.

In some embodiments, the search for a signal indicative of a GI is performed using traditional search methodologies. For example, in some embodiments, candidate signals indicative of a GI are identified based on sequence identity. In some embodiments, identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0 or later) of Altschul et al., J. Mol. Biol. 215:403-10, 1990. BLAST® protein searches can be performed, for example, with the XBLAST program, score=50, wordlength=3 to obtain amino acid sequences homologous to the proteins described herein. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

In some embodiments, candidate signals indicative of a GI exhibit at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a known gene encoding a signal indicative of a GI.

In some embodiments, signals of GIs are identified based on sequence similarity. Similarity of nucleic acid sequences and protein sequences can be assessed by a number of methods, including those known in the art, in accordance with the present disclosure.

Widely used similarity searching programs known to persons having skill in the art include: BLAST (Altschul S F, Madden T L, Schaffer A A, Zhang J, Zhang Z, Miller W, Lipman D J. Gapped BLAST and PSI-BLAST: a new generation of protein database search programs. Nucleic Acids Res. 1997;25:3389-3402; units 3.3 and 3.4), PSI-BLAST (Id.), SSEARCH (Smith T F, Waterman M S. Identification of common molecular subsequences. J. Mol. Biol. 1981; 147:195-197; Pearson WR. Searching protein sequence libraries: Comparison of the sensitivity and selectivity of the smith-waterman and fasta algorithms. Genomics. 1991; 11:635-650, unit 3.10), FASTA (Pearson W R, Lipman D J. Improved tools for biological sequence comparison. Proc. Natl. Acad. Sci. USA. 1988; 85:2444-2448 unit 3.9), and MUSCLE (Edgar RC. MUSCLE: multiple sequence alignment with high accuracy and high throughput. Nucleic Acids Res. 2004;32(5):1792-1797).

In some embodiments, signals of GIs exhibit at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity with a known gene encoding a signal of a GI. Thus, in some embodiments, the term “signal of a GI” or “GI Indicator” is understood to encompass homologs and orthologs thereof.

In some embodiments, signals of GIs are identified via predictive engines. In some embodiments, the predictive engines are machine learning models. In some embodiments the predictive engines are HMM models.

Persons having skill in the art will be familiar with the various public sources for EIMIM sequence models, and/or with methods of generating new machine learning models for conducting gene searches for signals indicative of a GI. For example, in some embodiments, the present disclosure teaches the use of TIGRFam or PFam EIMIM models to identify candidate insecticidal genes (e.g. to identify signals indicative of an IG, or additional indications of a insecticidal protein). These EIMMs are available for a wide range of types of proteins, and can be applied directly the digital metagenomic libraries of the present disclosure. In some embodiments, at least one signal indicative of a GI is identified using an HMM model trained on known sequences comprising the signal indicative of the GI.

TIGRFAM is a resource consisting of curated multiple sequence alignments, Hidden Markov Models (HMMs) for protein sequence classification, and associated information capable of searching for homologous proteins. Starting with release 10.0, TIGRFAMs models use EIMMER3, which provides good search speed as well and search sensitivity (Haft DH, et al., TIGRFAMs: a protein family resource for the functional identification of proteins. Nucleic acids research. 2001-01-01; 29.1: 41-3.)

Pfam similarly contains multiple alignments and hidden Markov model based profiles (HMM-profiles) of complete protein domains. The definition of domain boundaries, family members and alignment is done semi-automatically based on expert knowledge, sequence similarity, other protein family databases and the ability of BM M-profiles to correctly identify and align the members (Sonnhammer E L, Eddy SR, Birney E, Bateman A, Durbin R. Pfam: multiple sequence alignments and HMM-profiles of protein domains. Nucleic Acids Res. 1998; 26(1):320-322).

### GI Indicator Gene Search Output and Optional Filtering

In some embodiments, the present disclosure recites identification of signals indicative of a GI and additional indications of an insecticidal protein encoding gene. As discussed above, in some embodiments, these elements are identified via standard sequence homology searching tools based on sequence identity or sequence similarity. In some embodiments, these elements are identified via the use of machine learning models, as described in more detail, below.

In some embodiments, the signal indicative of a GI is a gene (e.g., invertase, endonuclease, etc). In some embodiments, the output from the gene homology searches for a signal indicative of a GI is a plurality of candidate GI indicators. In some embodiments, each candidate GI indicator gene sequence is associated with a confidence score related to the likelihood that the search model's prediction is accurate. Thus, candidate GI indicator gene sequences may be identified based on the confidence score assigned to the candidate sequence by the model (e.g., a machine learning model, e.g., an HMM).

In some embodiments, the present disclosure teaches keeping all predicted gene candidate sequences for the next workflow step. In some embodiments, the present disclosure teaches the use of pre-selected confidence cutoffs, so that only the hits with the best confidence proceed to subsequent steps of the analysis. The confidence score cutoff may vary based on the size of the database and other features of the particular implementation of the method. Alternatively, the method or system may employ other means for discriminating between candidate sequences and non-candidate sequences. In some embodiments, the candidate GI indicator gene sequences are ranked in order of highest confidence to lowest confidence by their confidence score and then a cutoff is employed to remove any sequences falling below a particular confidence threshold. For example, if the confidence score is an e-value, the candidate sequences may be ranked in order of ascending e-value: lowest e-value (highest confidence) to highest e-value (lowest confidence). Then, any sequences assigned an e-value above a selected threshold may be removed from the pool of candidate sequences. In some embodiments, the e-value is calculated using a BLAST or HIMIVI search. Analogously, if the confidence score is a bit score, the candidate sequences may be ranked in order of descending bit score: highest bit score (highest confidence) to lowest bit score (lowest confidence). In some embodiments, the bit score is calculated using a BLAST or HIMIVI search. Then, any sequences assigned a bit score below a selected threshold may be removed from the pool of candidate sequences.

In some embodiments, the present disclosure selects candidate sequences exhibiting at least 10, 20, 30, 40, 50, 60, 70, 80, 90, 100, 110, 120, 130, 140, 150, 160, 170, 180, 190, 200, 210, 220, 230, 240, 250, 260, 270 280, 290, or 300 bitscore.

In some embodiments, following identification of the candidate GI indicator gene sequences from the sequence database, the candidate sequences are filtered to remove GI sequences that are less likely to perform a function of a signal indicative of an GI (for example, a function of a gene encoding an integrase, a gene encoding a transposase, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage, or a phage gene). In some embodiments, the candidate sequences are filtered based on their evaluation using one or more second “control” predictive models. The number of control predictive models employed may depend on the situation, the type of candidate GI indicator gene, the availability of relevant data, and other such features. In some embodiments, the number of control predictive models is between 1 and 100,000. In some embodiments, the number of control predictive models is at least 1, at least 10, at least 100, at least 1,000, at least 10,000, or at least 100,000.

In some embodiments, the candidate GI indicator gene sequences are evaluated by a first predictive model that determines the likelihood that the sequence performs the function of the target GI, e.g., by assigning a confidence score; then, the candidate sequences are evaluated by a second predictive model or models that determine the likelihood that the sequence performs a different function, e.g., by assigning a confidence score. The relative likelihoods of the candidate GI indicator gene sequence performing the target protein or target gene function (e.g., the function of a gene encoding an integrase, a gene encoding a transposase, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage, or a phage gene) or another function are then compared. In some embodiments, each candidate sequence is assigned a “target GI indicator gene confidence score” generated by the first predictive model and a “best match confidence score”, wherein the best match confidence score is the best confidence score generated by a second predictive model evaluating the likelihood that the candidate sequence performs a different function than the target protein or target gene function. For example, if 500 control predictive models are employed to determine whether or not the sequence is likely to encode a protein or gene performing a function other than the target protein or target gene function, the “best match confidence score” would be the best confidence score (e.g., highest bit score, lowest e-value) generated by any one of the 500 control predictive models.

Thus, in some embodiments, the target protein or target gene confidence score and the best match confidence score are compared. In some embodiments, the log of the target protein or target gene e-value and the log of the best match (e.g., from the second predictive machine learning model) e-value are compared. In some embodiments, the target protein or target gene bit score and the best match bit score are compared. In some embodiments, a threshold is established for the relative likelihood of performing the target protein or target gene function.

The number of control predictive machine learning models employed is not numerically limited, but is based on the ability to generate and/or availability of control models, such as those which may be generated based on the identification of orthology groups other than those to which the target protein or target gene belongs. In some embodiments, at least one secondary model is employed. In some embodiments, at least 5, 10, 50, 100, 200, 300, 400, 500, 600, 700, 800, 900, 1,000, or 10,000 control models are employed.

In some embodiments, candidate GI indicator gene sequences are only retained if the likelihood of performing the target protein or target gene function is greater than the likelihood of performing a different protein function. In some embodiments, candidate GI indicator gene sequences are only retained if the likelihood of performing the GI function is greater than or approximately equal to the likelihood of performing a different protein function. In some embodiments, the candidate GI indicator gene sequence is retained if the relative likelihood of performing the target GI gene function falls within a certain confidence interval. In some embodiments, the candidate GI indicator gene sequence is retained if the relative likelihood of performing the target GI function exceeds a certain threshold value. In some embodiments, a candidate GI indicator gene sequence is retained if it meets the following criteria (or the equivalent for a target GI):

$$\\ \frac{candidate GI indicator bit score}{best match bit score} or$$

$$\\ \frac{log ( candidate GI E value )}{log ( best match E value )} > threshold value .$$

In some embodiments, the best match E value or best match bit score is the best confidence score out of the control predictive models. In other embodiments, the best match is the best confidence score out of all tested predictive models, including the candidate GI confidence score. In this second embodiment, if the candidate GI indicator gene confidence score (e.g. bit score or E value) is the best match, then the ratio is 1. In other embodiments, in which the best match confidence score is selected from amongst the control predictive models, the ratio can exceed 1.

The threshold value for retaining a candidate GI indicator gene sequence may be modified based on the desired confidence range. In some embodiments the threshold value is between 0.1 and 0.99. In some embodiments, the threshold value is between 0.5 and 0.99. In some embodiments, the threshold value is 0.1, 0.2, 0.3, 0.4, 0.5, 0.6, 0.7, 0.8, or 0.9. In some embodiments, the threshold value is 0.5, 0.55, 0.6, 0.65, 0.7, 0.75, 0.8, 0.85, 0.9, or 0.95.

The threshold calculations above are illustrative, but in no way exhaustive. Persons having skill in the art will recognize how to apply various threshold cutoffs depending on how their confidence scores are calculated. For example, if the confidence score is such that a lower score indicates greater confidence, then a sequence may be retained if the ratio of the target protein or target gene confidence score to the best match confidence score is lower than a certain threshold value.

In some embodiments each of the output candidate GI indicator genes will be associated with a longer DNA sequence (i.e., each candidate GI gene will be contained within a longer assembled DNA sequence within the digital metagenomic library). In some embodiments, the present disclosure teaches filtering out candidate GIs that are contained within assembled DNA sequences that are less than 10 kb, 9 kb, 8 kb, 7 kb, 6 kb, 5 kb, 4 kb, 3 kb, 2 kb, or 1 kb in length. In some embodiments, the sequence length cutoff is made based on the expected size of the GI. If the expected insecticidal protein is expected to be comprised within a GI of at least 30 kb, it may not be relevant to further process candidate GIs of less than 10 kb in length.

In some embodiments, the outputs of the GI searches may also be filtered based on the predicted taxonomy of the assembled DNA sequence. Thus, if the goal is to identify insecticidal proteins from Actinobacteria, sequences which had been identified as belonging to other genus/species can be filtered out prior to subsequent workflow steps.

In some embodiments, the outputs of the GI gene searches may also be filtered to remove duplicates, or highly related sequences. In some embodiments the GI results may also be filtered remove partial sequences.

In some embodiments, the outputs of the GI searching can be prioritized based on the each candidate GI indicator genes sequence's homology to a corresponding target GI from another organism. Thus, in some embodiments, candidate GI indicator gene sequences are compared to a known database using BLAST, to determine whether the strongest matches are identified by blast as homologs to the desired candidate sequence.

In some embodiments, DNA digital sequences from the digital metagenomics library that are identified as comprising a candidate GI indicator gene, and which (optionally) further survive the filtering steps described above and herein referred to as “signal-associated multi-gene cluster digital feature sets.”

Unfiltered sequences are permitted to proceed along the presently disclosed workflow.

The machine learning searching strategies of the present disclosure have been described above in the context of identifying signals indicative of a GI. Persons having skill in the art will understand however that the same strategies are also applicable to the recited searches for additional indications of an insecticidal protein encoding gene.

Screening the Digital Metagenomics Library for Additional Indications of an Insecticidal Protein (in silico)

In some embodiments, the methods of the disclosure comprise screening the GI-associated digital feature sets for additional indications of an insecticidal protein encoding gene. In some embodiments, this is an in silico screening step. In some embodiments, the additional indications of an insecticidal protein encoding gene comprises the presence of a pathogenicity factor within an open reading frame of the GI-associated digital feature sets. In some embodiments, at least one pathogenicity factor is identified using an HMIVI model trained on known sequences comprising the pathogenicity factor.

In some embodiments, the additional indications of an insecticidal protein encoding gene comprises the presence of a pathogenicity factor within an open reading frame of the GI-associated digital feature set, said pathogenicity factor selected from the group consisting of: predicted molecular weight for an encoded protein, predicted hydrophobicity for an encoded protein, presence or lack of transmembrane domain within an encoded protein, presence or lack of other domains of interest (e.g. DUF domain) within an encoded protein, sequence similarity to known insecticidal proteins of an encoded protein, sequence similarity to other proteins found in known GIs, and predicted taxonomy of a sequence within the digital feature sets.

In some embodiments, the pathogenicity factor is molecular weight. In some embodiments, the molecular weight of an insecticidal protein within an open reading frame of the GI-associated digital feature sets ranges from about 10 kDa to about 200 kDa, for example, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 130 kDa, about 135 kDa, about 140 kDa, about 145 kDa, about 150 kDa, about 155 kDa, about 160 kDa, about 165 kDa, about 170 kDa, about 175 kDa, about 180 kDa, about 185 kDa, about 190 kDa, about 195 kDa, or about 200 kDa, including all ranges and subranges therebetween.

In some embodiments, the pathogenicity factor is predicted hydrophobicity of a putative insecticidal protein within an open reading frame of the GI-associated digital feature sets. In some embodiments, the hydrophobicity of a candidate insecticidal protein is identified using a hydrophobicity scale. In some embodiments, the hydrophobicity scale is selected from the group consisting of CHOTA, CHOC760103, CHOTH, PONNU, Sweet and Eisenberg, KIDER, ROSEB, Welling, Rao and Argos, FASG890101, GIBRA, BULDG reverse, LEVM760101 reverse, Bishop reverse, Wimley reverse, Welling reverse, ENGD860101 reverse, ROSEA reverse, WILM950103 reverse, Kuh1950101 reverse, PRAM900101 reverse, FASG890101 reverse, GUYH850101 reverse, KIDA850101 reverse, ROSM880102 reverse, ROSM880103 reverse, ROSM880101 reverse, WOLR810101 reverse, BULDG, MANP780101, VHEG790101, JANJ790102, JANIN, WOLR790101, PONP800101, PONP800102, PONP800103, PONP800104, PONP800105,

PONP800106, Wilson, ARGP820101, FAUJ830101, FAUCH, MIYS850101, ENGEL, ROSEM, JACWH, PARJ860101, Cowan Whittacker, ROSM880101, ROSM880102, COWR900101, BLAS910101, CASSI, CIDH920101, CIDH920105, CIDBB, CIDA+, CIDAB, PONG1, PONG2, PONG3, PONP030101, WILM950101, WILM950102, WILM950104, Wimley, Bishop, NADH010101, NADH010102, NADH010103, NADH010104, NADH010105, NADH010106, and NADH010107. These scales are described in detail in the following documents which are incorporated by reference herein in their entirety:
(a) Simm et al. Biol Res. 2016; 49: 31.
(b) Eisenberg D. Annu Rev Biochem. 1984; 1984(53):595-623.
(c) Guy H R. Amino acid side-chain partition energies and distribution of residues in soluble proteins. Biophys J. 1985; 1985(47):61-70.
(d) Rose G D, Geselowitz A R, Lesser G J, Lee R H, Zehfus M H. Hydrophobicity of amino acid residues in globular proteins. Science. 1985; 1985(229):834-838.
(e) Fasman G D. Prediction of protein structure and the principles of protein conformation. New York: Plenum; 1989. p. 789.
(f) Chothia C. The nature of the accessible and buried surfaces in proteins. J Mol Biol. 1976; 1976(105):1-12.
(g) Tanaka S, Scheraga H A. Statistical mechanical treatment of protein conformation. I. Conformational properties of amino acids in proteins. Macromolecules. 1976; 1976(9):142-159.
(h) Welling G W, Weijer W J, van der Zee R, Welling-Wester S. Prediction of sequential antigenic regions in proteins. FEBS Lett. 1985; 1985(188):215-218.
(i) Mohana Rao J K, Argos P. A conformational preference parameter to predict helices in integral membrane proteins. Biochim Biophys Acta. 1986; 1986(869):197-214.
(j) Bull H B, Breese K. Surface tension of amino acid solutions: a hydrophobicity scale of the amino acid residues. Arch Biochem Biophys. 1974; 1974(161):665-670.
(k) Levitt M. A simplified representation of protein conformations for rapid simulation of protein folding. J Mol Biol. 1976; 1976(104):59-107.
(1) Bishop C M, Walkenhorst W F, Wimley W C. Folding of beta-sheets in membranes: specificity and promiscuity in peptide model systems. J Mol Biol. 2001; 2001(309):975-988.
(m) Wimley W C, White S H. Experimentally determined hydrophobicity scale for proteins at membrane interfaces. Nat Struct Biol. 1996; 1996(3):842-848.
(n) Kuhn L A, Swanson C A, Pique M E, Tainer J A, Getzoff ED. Atomic and residue hydrophilicity in the context of folded protein structures. Proteins. 1995; 1995(23):536-547.
(o) Prabhakaran M. The distribution of physical, chemical and conformational properties in signal and nascent peptides. Biochem J. 1990; 1990(269):691-696.
(p) Roseman M A. Hydrophilicity of polar amino acid side-chains is markedly reduced by flanking peptide bonds. J Mol Biol. 1988; 1988(200):513-522.
(q) Jones D D. Amino acid properties and side-chain orientation in proteins: a cross correlation approach. J Theor Biol. 1975; 1975(50):167-183.
(r) Wilce M C J, Aguilar M I, Hearn M T W. Physicochemical basis of amino acid hydrophobicity scales: evaluation of four new scales of amino acid hydrophobicity coefficients derived from RP-HPLC of peptides. Anal Chem. 1995; 1995(67):1210-1219.
(s) Ponnuswamy P K, Prabhakaran M, Manavalan P. Hydrophobic packing and spatial arrangement of amino acid residues in globular proteins. Biochim Biophys Acta. 1980; 1980(623):301-316.
(t) Cid H, Bunster M, Canales M, Gazitna F. Hydrophobicity and structural classes in proteins. Protein Eng. 1992; 1992(5):373-375.
(u) Fauchere J-L, PliSka V. Hydrophobic parameters H of amino-acid side chains from the partitioning of N-acetyl-amino-acid amides. Eur J Med Chem. 1983; 18:369-375.
(v) Zviling M, Leonov H, Arkin I T. Genetic algorithm-based optimization of hydrophobicity tables. Bioinformatics. 2005; 2005(21):2651-2656.
(w) Juretie D. Protein secondary structure conformations and associated hydrophobicity scales. J Math Chem. 1993; 1993(14):35-45.
(x) Zimmerman J M, Eliezer N, Simha R. The characterization of amino acid sequences in proteins by statistical methods. J Theor Biol. 1968; 1968(21):170-201.
(y) Nozaki Y, Tanford C. The solubility of amino acids and two glycine peptides in aqueous ethanol and dioxane solutions. Establishment of a hydrophobicity scale. J Biol Chem. 1971; 1971(246):2211-2217.
(z) Kyte J, Doolittle R F. A simple method for displaying the hydropathic character of a protein. J Mol Biol. 1982; 1982(157):105-132.
(aa) Engelman D M, Steitz T A, Goldman A. Identifying nonpolar transbilayer helices in amino acid sequences of membrane proteins. Ann Rev of Biophys Biophys Chem. 1986; 1986(15):321-353.
(bb) Naderi-Manesh H, Sadeghi M, Arab S, Moosavi Movahedi A A. Prediction of protein surface accessibility with information theory. Proteins. 2001; 2001(42):452-459.
(cc) Cornette J L, Cease K B, Margalit H, Spouge J L, Berzofsky J A, DeLisi C. Hydrophobicity scales and computational techniques for detecting amphipathic structures in proteins. J Mol Biol. 1987; 1987(195):659-685.
(dd) Sweet R M, Eisenberg D. Correlation of sequence hydrophobicities measures similarity in three-dimensional protein structure. J Mol Biol. 1983; 1983(171):479-488.
(ee) Wolfenden R V, Cullis P M, Southgate C C. Water, protein folding, and the genetic code. Science. 1979; 1979(206):575-577.
(ff) Manavalan P, Ponnuswamy P K. Hydrophobic character of amino acid residues in globular proteins. Nature. 1978; 1978(275):673-674.
(gg) von Heijne G, Blomberg C. Trans-membrane translocation of proteins. The direct transfer model. Eur J Biochem. 1979; 1979(97):175-181.
(hh) Janin J. Surface and inside volumes in globular proteins. Nature. 1979; 1979(277):491-492.
(ii) Wilson K J, Honegger A, Stotzel R P, Hughes G J. The behaviour of peptides on reverse-phase supports during high-pressure liquid chromatography. Biochem J. 1981; 1981(199):31-41.
(jj) Argos P, Rao J K, Hargrave P A. Structural prediction of membrane-bound proteins. Eur J Biochem. 1982; 1982(128):565-575.
(kk) Miyazawa S, Jernigan R L. Estimation of effective interresidue contact energies from protein crystal structures: quasi-chemical approximation. Macromolecules. 1985; 1985(18):534-552.
(11) Jacobs R E, White S H. The nature of the hydrophobic binding of small peptides at the bilayer interface: implications for the insertion of transbilayer helices. Biochemistry. 1989; 1989(28):3421-3437.
(mm) Parker J M, Guo D, Hodges R S. New hydrophilicity scale derived from high-performance liquid chromatography peptide retention data: correlation of predicted surface residues with antigenicity and X-ray-derived accessible sites. Biochemistry. 1986; 1986(25):5425-5432.
(nn) Cowan R, Whittaker R G. Hydrophobicity indices for amino acid residues as determined by high-performance liquid chromatography. Pept Res. 1990; 1990(3):75-80.
(oo) Black S D, Mould D R. Development of hydrophobicity parameters to analyze proteins which bear post- or cotranslational modifications. Anal Biochem. 1991; 1991(193):72-82.
(pp) Casari G, Sippl M J. Structure-derived hydrophobic potential. Hydrophobic potential derived from X-ray structures of globular proteins is able to identify native folds. J Mol Biol. 1992; 1992(224):725-732. doi: 10.1016/0022-2836(92)90556-Y.
(qq) Ponnuswamy P K, Gromiha M M. Prediction of transmembrane helices from hydrophobic characteristics of proteins. Int J Pept Protein Res. 1993; 1993(42):326-341.

In some embodiments, the pathogenicity factor is the presence of a transmembrane domain within an open reading frame of the GI-associated digital feature sets. In some embodiments, an insecticidal domain comprises a transmembrane domain. In some embodiments, an insecticidal protein lacks a transmembrane domain. In some embodiments, the presence of a transmembrane domain is identified using a hydrophobicity scale described herein.

In some embodiments, the pathogenicity factor is sequence identity of an ORF within the GI-associated digital feature sets with a known insecticidal protein.

In some embodiments, the pathogenicity factor is the presence or lack of specific domains of interest within an open reading frame of the GI-associated digital feature sets. For example, in some embodiments, insecticidal proteins comprise one or more of the following domains: endotoxin, ETX-MTX, aerolysin, aegerolysin, bacillus toxin, ricinB, CDToxin, or Tc toxin domains.

In some embodiments, the pathogenicity factor is sequence similarity/homology of an open reading frame of the GI-associated digital feature sets to a known insecticidal protein. In some embodiments, the pathogenicity factor is sequence similarity/homology of other sequences within the GI-associated digital feature set to other genes found within known GIs. For example, in some embodiments, the additional indications of an insecticidal protein encoding gene is the presence of surrounding genes selected from the group consisting of: a gene encoding an integrase, a gene encoding a transposase, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage gene, or a phage gene.

In some embodiments, the pathogenicity factor is the identification of taxonomy associated with insecticidal proteins. In some embodiments, the insecticidal protein gene is identified as comprising to a genus selected from the group consisting of Bacillus, Pseudomonas, Xenorhabdus, Photorhabdus, Chromobacteria, Entomophthorales, and Hypocreales.

### Screening the Digital Metagenomics Library for Additional Indications of an Insecticidal Protein (Experimental)

In some embodiments, the screening for additional indications of an insecticidal protein comprises experimentally confirming insecticidal function for a protein encoded by an open reading frame within the GI-associated digital feature sets.

In some embodiments, experimental confirmation of insecticidal function is conducted by expressing the protein encoded by the open reading frame within the GI-associated digital feature sets in a microbial culture, and exposing said microbial culture to an insect. In some embodiments, the microbial culture is lysed before the insect is exposed to it. In some embodiments, after lysis the expressed protein is purified. In some embodiments the ORF from the GI-associated digital feature set is expressed in a cell-free translation system.

In some embodiments, experimental confirmation of insecticidal function is conducted by expressing the protein encoded by the open reading frame within the GI-associated digital feature sets in a microbial culture, and exposing the expressed candidate insecticidal protein to an insect. In some embodiments, the expressed candidate insecticidal protein is purified prior to exposure to an insect. In some embodiments, the insect is exposed to the microbial culture expressing the candidate insecticidal protein.

In some embodiments, experimental confirmation of insecticidal function is performed according to the methods of Ruffner et al. BMC Genomics (2015) 16:609. This document is incorporated by reference herein in its entirety.

In some embodiments, the expressed candidate insecticidal protein inhibits the growth or development of the insect.

In some embodiments, the expressed candidate insecticidal protein kills the insect.

In some embodiments, the candidate insecticidal protein is part of a multi-gene toxin complex.

Annotating Genes Within GIs

In some embodiments, carrying out the steps of the presently-disclosed methods comprises the sub step of annotating genes/sequences within the digital metagenomics library or within the GI-associated digital feature sets. In some embodiments, the annotation of the digital metagenomics library or within the GI-associated digital feature sets is done based on sequence or structural homology of genes to known genes using one or more annotation engines.

Persons having skill in the art will be familiar with the various other gene annotation tools compatible with the workflows of the present disclosure. A non-limiting list of annotation tools is provided as Table 2 below.

TABLE 2
Non-limiting List of Sequence Annotation Tools

| Name | Can be Used For | Algorithm | References |
|---|---|---|---|
| GeneMark | Archaea, | hidden Markov | Besemer J. and Borodovsky M. |
| | Metagenomes, | model | Nucleic Acids Research, 2005, Vol. |
| | Eukaryotes, Viruses, | | 33, Web Server Issue, pp. W451-454 |
| | Phages, Plasmids, | | |
| | EST and cDNA | | |
| GeneHacker | Microbial genomes | Markov model | Yada. T, Hirosawa. M DNA Res., 3, |
| | | | 335-361 (1996). Syst. Mol. Biol. |
| | | | pp. 252-260 (1996). Syst. Mol. Biol. |
| | | | pp. 354-357 (1997). |
| GeneWalker | Human | Hidden Markov | |
| | | model | |
| HMMgene (v. 1.1) | vertebrate and C. | Hidden Markov | A. Krogh: In Proc. of Fifth Int. Conf. |
| | elegans | model | on Intelligent Systems for Molecular |
| | | | Biology, ed. Gaasterland, T. et al., |
| | | | Menlo Park, CA: AAAI Press, 1997, |
| | | | pp. 179-186. |
| Chemgenome2.0 | Prokaryotes | Ab-initio Method | Poonam Singhal, B. Jayaram, Surjit |
| | | | B. Dixit and David L. Beveridge. |
| | | | Prokaryotic Gene Finding based on |
| | | | Physicochemical Characteristics of |
| | | | Codons Calculated from Molecular |
| | | | Dynamics Simulations. Biophysical |
| | | | Journal, 2008, Volume: 94 Issue: 11, |
| | | | 4173-4183] |
| Softberry Server | Bacteria, Viruses | HMM and | Solovyev V. V., Salamov A. A., |
| | and eukaryotes | similarity based | Lawrence C. B. (Nucl. Acids |
| | | searches | Res., 1994, 22, 24, 5156-5163). |
| Gene ID | Animal, Human, | Neural Network | Blanco et. al., Genome Research |
| | Plants fungus, | | 6(4): 511-515 (2000). |
| | Protists | | |
| GenScan | Vertebrates, | Ab-inito Method | Burge and Karlin (1998) Curr. Opin. |
| | Arabidopsis, Maize | | Struct. Biol. 8, 346-354. |
| GenomeThreader | Plants | Similarity-based | Gremme et al Information and |
| | | gene prediction | Software Technology, 47(15): 965- |
| | | program where | 978, 2005 |
| | | additional | |
| | | cDNA/EST and/or | |
| | | protein sequences | |
| | | are used to predict | |
| | | gene structures via | |
| | | spliced alignments | |
| JIGSAW(formerly | Eukaryotes | multiple sources of | Allen et al. Genome Biology 2007, |
| “Combiner”) | | evidence (output | 7(Suppl): S9.; Allen and Salzberg |
| | | from gene finders, | Bioinformatics 21(18): 3596-3603, |
| | | splice site | 2005; Allen et al. Genome Research, |
| | | prediction programs | 14(1), 2004. |
| | | and sequence | |
| | | alignments to | |
| | | predict gene | |
| | | models) | |
| GlimmerHMM | Eukaryotes | GlimmerHMM is | Majoros et al. Bioinformatics 20 |
| | | based on a | 2878-2879, 2004 |
| | | Generalized Hidden | |
| | | Markov Model | |
| | | (GHMM). | |
| | | Although the gene | |
| | | finder conforms to | |
| | | the overall | |
| | | mathematical | |
| | | framework of a | |
| | | GHMM, | |
| | | additionally it | |
| | | incorporates splice | |
| | | site models adapted | |
| | | from the | |
| | | GeneSplicer | |
| | | program and a | |
| | | decision tree | |
| | | adapted from | |
| | | GlimmerM. It also | |
| | | utilizes Interpolated | |
| | | Markov Models for | |
| | | the coding and | |
| | | noncoding models | |
| | | Currently, | |
| | | GlimmerHMM's | |
| | | GHMM structure | |
| | | includes introns of | |
| | | each phase, | |
| | | intergenic regions, | |
| | | and four types of | |
| | | exons (initial, | |
| | | internal, final, and | |
| | | single). | |
| GenZilla | Eukaryotes | GeneZilla is based | GeneZilla (formerly “TIGRscan”) is |
| | | on the Generalized | briefly described in: Majoros W, et |
| | | Hidden Markov | al. (2004) Bioinformatics 20, 2878- |
| | | Model (GHMM). It | 2879 The novel decoding algorithm |
| | | evolved out of the | used by GeneZilla is described in: |
| | | ab initio eukaryotic | Majoros W. et al. (2005) BMC |
| | | gene finder | Bioinformatics 5: 616. |
| | | TIGRscan, which | |
| | | was developed at | |
| | | The Institute for | |
| | | Genomic Research. | |
| Twinscan/N- | | TWINSCAN | TWINSCAN: Gross and Brent. J |
| SCAN (Ver 4.1.2) | | extends the | Comput Biol. 2006 March; 13(2): 379- |
| | | probability model | 93. Korf I, N-SCAN: Flicek et al |
| | | of GENSCAN, | Bioinformatics. 2001; 17 Suppl |
| | | allowing it to | 1: S140-8. |
| | | exploit homology | |
| | | between two related | |
| | | genomes. Separate | |
| | | probability models | |
| | | are used for | |
| | | conservation in | |
| | | exons, introns, | |
| | | splice sites, and | |
| | | UTRs, reflecting the | |
| | | differences among | |
| | | their patterns of | |
| | | evolutionary | |
| | | conservation. | |
| Manatee | prokaryotic and | Manatee is a web- | NA |
| | eukaryotic genomes | based gene | |
| | | evaluation and | |
| | | genome annotation | |
| | | tool that can view, | |
| | | modify, and store | |
| | | annotation for | |
| | | prokaryotic and | |
| | | eukaryotic | |
| | | genomes. The | |
| | | Manatee interface | |
| | | allows biologists to | |
| | | quickly identify | |
| | | genes and make | |
| | | high quality | |
| | | functional | |
| | | assignments using a | |
| | | multitude of | |
| | | genome analyses | |
| | | tools. These tools | |
| | | consist of, but are | |
| | | not limited to GO | |
| | | classifications, BER | |
| | | and blast search | |
| | | data, paralogous | |
| | | families, and | |
| | | annotation suggestions | |
| | | generated from | |
| | | automated analysis. | |
| EvoGene | NA | alignment of | Pedersen and Hein. Bioinformatics |
| | | multiple genomic | (in press) |
| | | sequences | |
| CRITICA(Coding | Prokaryotic | CRITICA combines | Badger and Olsen. Molecular |
| Region | | traditional | Biology and Evolution, 16(4): 512- |
| Identification Tool | | approaches to the | 524. 1999. |
| Invoking | | problem with a | |
| Comparative | | novel comparative | |
| Analysis) | | analysis. If, in a | |
| | | nucleotide | |
| | | alignment, a pair of | |
| | | ORFs can be found | |
| | | in which the | |
| | | conceptual | |
| | | translated products | |
| | | are more conserved | |
| | | than would be | |
| | | expected from the | |
| | | amount of | |
| | | conservation at the | |
| | | nucleotide level, | |
| | | this is evolutionary | |
| | | evidence that the | |
| | | DNA sequences are | |
| | | protein coding. | |
| | | Regions found by | |
| | | this method are used | |
| | | to generate | |
| | | traditional dicodon | |
| | | frequencies for | |
| | | further analysis and | |
| | | give the prediction | |
| | | about a probable | |
| | | protein coding | |
| | | region. | |
| sgp2 | | Sgp2 predict genes | Parra et al. Genome Research |
| | | by comparing | 13(1): 108-117 (2003) |
| | | anonymous | |
| | | genomic sequences | |
| | | from two different | |
| | | species. Further it | |
| | | combines tblastx, a | |
| | | sequence similarity | |
| | | search program, | |
| | | with geneid, an “ab | |
| | | initio” gene | |
| | | prediction program. | |
| Phat | Eukaryotes (Homo | Phat is a HMM- | Unpublished |
| | sapiens, | based genefinder, | |
| | Plasmodium | originally | |
| | falciparum, | developed for | |
| | Plasmodium vivax) | genefinding in | |
| | | Plasmodium | |
| | | falciparum. | |
| EuGene | Eukaryotes | Eugene exploit | LNCS 2066, pp. 111-125, 2001 |
| | | probabilistic | |
| | | models like Markov | |
| | | models for | |
| | | discriminating | |
| | | coding from non- | |
| | | coding sequences or | |
| | | to discriminate | |
| | | effective splice sites | |
| | | from false splice | |
| | | sites (using various | |
| | | mathematical models). | |
| AUGUSTUS | Eukaryotic genomic | It allows to use | Stanke and Waack (2003) |
| | sequences | protein homology | Bioinformatics, Vol. 19, Suppl. 2, |
| | | information and | pages ii215-ii225 |
| | | travel in the | |
| | | prediction. | |

### Construction of HMMs

Several of the homology searches described in this document can be conducted via HMM searches. In some embodiments, at least one of the GI indicators is identified using an HMM model trained on known sequences comprising the GI indicator. In some embodiments, at least one of the pathogenicity factor(s) is identified using an HMM model trained on known sequences comprising the pathogenicity factor. In some embodiments, In some embodiments, the HMM searches are based on existing HMM models, such as those available in Pfam and TIGRfam. In other embodiments, the present disclosure teaches methods of constructing new HMMs designed to search for candidate homolog genes. Methods of constructing custom HMMs for searching candidate homolog genes are discussed in more detail below.

The present disclosure, in some embodiments, provides methods and systems making use of Hidden Markov Models (HMMs) for the prediction of candidate homolog genes (e.g., candidate GI genes, or for the purpose of annotation through homology to a gene with a known function). For the sake of simplicity however, sections below will generically refer to use of HMMs to identify homologs to a target gene/protein.

The following provides an exemplary workflow for generating an HMM for use in the present methods and systems. In some embodiments, an HMM generation workflow comprises the following steps: 1) Identify sequences to be used in a training data set corresponding to the target GI gene; 2) Align the sequences; 3) Evaluate the alignment; 4) Generate the HMM predictive machine learning model from the multiple sequence alignment; 5) Evaluate the HMM.

Each of these exemplary steps is elaborated on herein.

1. Identify Sequences to be Used in Training Data Set

To construct an HMM to make predictions about whether or not a given sequence is a homolog to a target gene/protein, it is necessary to have a set of target sequences (at least one) that exhibits the desired properties (i.e., has been determined to belong to an annotation category of interest, such as belonging to a genus of target GI genes). This is the initial training data set that will be used to train the machine learning model (e.g., HMM) in the present methods and systems: the data set comprises input genetic data (nucleic acid and/or amino acid sequences) and output phenotypical data (that the sequence performs the desired function). The list may be generated from either an existing orthology group (e.g., a KEGG orthology group) identified as having the desired function, or by identifying a sequence performing the desired function in Uniprot and finding homologs of that sequence. In some embodiments, the list may be compiled from a publicly available sequence database. In some embodiments, the list may be compiled from a proprietary database. In some embodiments, the list may be compiled from a commercial database. In some embodiments, the list may be compiled from empirical data, such as validation experiments.

In some embodiments, the present disclosure teaches that the predictive ability of the HMM can be improved by providing the model with diverse sequences encoding proteins performing the desired function, i.e., the target protein function, or diverse sequences encoding genes performing the desired function, i.e., the target gene function. A very similar sequence set may train the HMM to identify similar sequences, similar to BLAST. Diverse sequences allow the HMM to capture which positions (e.g., amino acids) can vary and which are important to conserve. In some embodiments, it is desirable to include as many sequences as possible that are reasonably expected to perform the desired target function.

In some embodiments, the present disclosure teaches that the sequences in the training data set should share one or more sequence features. If sequences in the training data set do not share any common sequence features, they are likely not orthologs and should be excluded from the training data set. In some embodiments, the present disclosure teaches the creation of a primary HMM trained solely on high confidence training data sets, and a separate HMM trained on sequences selected with more lenient guidelines, such as outlier sequences that are believed to have the desired function, but do not share many of the sequence features present within the rest of the training data set. This permits the users to the analysis results with high vs. low confidence training data, providing flexibility for any downstream analysis.

For the purposes of illustration, the guidance for the identification of an initial training data set of sequences is applied to the target protein integrase, which could be identified as a signal indicative of a GI. These steps may be followed by an individual or may be programmed into software as a part of a method or system. To find an initial sequence training data set for the target protein integrase, one may start by looking for an existing orthology group annotated with the desired function, e.g., as follows:

Search KEGG orthology database for the desired term (www.genome.jp/dbget-bin/www_bfind_sub?mode=bfind&max_hit=1000&dbkey=orthology&keywords=integrase).

Select the KEGG Orthology link.

Scroll down to Genes and select the Uniprot link to get a list of Uniprot IDs for this function.

Cut and paste the list of Uniprot IDs into Excel to get a column of the IDs separate from the descriptions.

Go to Retrieve/ID at Uniprot.

Paste the set of Uniprot IDs retrieved in step (e). This will return a list of Uniprot entries. Select the download link to retrieve a list sequences of these entries in FASTA format.

It is also possible to compile an initial training data set by searching Uniprot for a desired sequence, e.g., as follows:

Search UniprotKB for a protein performing the function of the target protein in any organism, e.g., an organism of interest. For this example, the search begins with the exemplary integrase found at www.uniprot.org/uniprot/Q76353.

In the upper left corner, there is a button to do a BLAST search of this sequence against the full UniprotKB. Click this, and select the advanced option.

Set Threshold to 0.1 and Hits to 1000; this will provide a large number of hits while removing very different sequences. Then run the search. It will take a few minutes to complete the search.

Click the download link to download all sequences as a FASTA file.

2. Align the Sequences

The sequences accumulated in step 1 may be aligned using any available multiple sequence alignment tool. Multiple sequence alignment tools include Clustal Omega, EMBOSS Cons, Kalign, MAFFT, MUSCLE, MView, T-Coffee, and WebPRANK, among others. For the purposes of this illustrative example, Clustal Omega is employed. Clustal Omega may be installed on a computer and run from the command line, e.g., with the following prompt:

$ clustalo -infile=uniprot-list.fasta -type=protein -output=fasta -outfile=aligned.fasta

3. Evaluate the Alignment (Optional)

The multiple sequence alignment performed in step 2 may be evaluated and filtered for poor matches. As described in the foregoing, sequences that do not share sequence features are likely not in the same orthology group and may be detrimental to the quality of the HMM.

For assisting in the evaluation of the alignment, exemplary in-browser alignment tools are //msa.biojs.net/ and //github.com/veidenberg/wasabi. Both can be downloaded and run locally.

Sequences that do not match the rest of the training data set may be removed from the training data set before proceeding to the next step. Such sequences may be removed in an automated fashion based on objective criteria of the quality of the alignment, such as not possessing one or more sequence features common to most other members of the orthology group. In some embodiments, sequences that do not match the orthology group may be removed by other means, e.g., visual inspection.

4. Generate the HMM Predictive Machine Learning Model Based on the Training Data Set

The HMM can be generated by any HMM building software. Exemplary software may be found at, or adapted from: mallet. cs.umass.edu; www.cs.ubc.va/˜murphyk/Software/HMM/hmm.html; cran. r-project.org/web/packages/HNIM/index.html; www.qub.buffalo.edu; //ccb.jhu.edu/software/glimmerhmm/. In some embodiments, the HMMER tool is employed.

For the purposes of this illustrative example, EIMMbuild is used and may be downloaded and run locally with the following command: $ hmmbuild test.hmm aligned.fasta

5. Evaluate the HMM (Optional)

To evaluate the HMM generated in step 4, it may be run on an annotated database to evaluate its ability to correctly recognize sequences. In this illustrative example, the HMM is used to query the SwissProt database, for which all annotations are presumed to be true. The results of this test run may be checked to see if the annotations of the search result match the function the HMM should represent.

With a fasta file (or files) of a search database of protein sequences (e.g., protein db.fasta), the following command can be run to get an output file of HMM matches with a corresponding E-value: $ hmmsearch -A 0 --cpu 8 -E 1e-20 --noali --notextw test.hmm protein_db.fasta>hmm.out

This command can also be used on the translated proteome of a genome to find all hits matching a functional motif.

The various options in this command correspond to the following:
-A 0: do not save multiple alignment of all hits to a file
--cpu 8: use 8 parallel CPU workers for multithreads
-E 1e-20 : report sequences <=1e-20 e-value threshold in output
--noali: don't output alignments, so output is smaller
--notextw: unlimit ASCII text output line width

In some embodiments, custom-built EIMMs built according to this and other known methods can be used to establish homology for any of the workflow steps of this disclosure (e.g., identifying candidate GIs, or annotating genes).

### EXAMPLES

### Example 1

### Preparation of Metagenomic Libraries

Collection: Approximately 1 kg of soil sample from a private field was collected and rocks, branches, and other non-soil matter were removed by passing the soil through a 2 mm wire sieve. DNA was extracted from ˜250 g of soil by first adding 300 mL of a CTAB-based lysis buffer (100 mM Tris-HCl, 100 mM EDTA, 1.5 M NaCl, 1% (w/v) CTAB, 2% (w/v) SDS, pH 8.0), followed by incubation at 70° C. for 2h with consistent inversion to mix. The sample was centrifuged at 4,000 g for 20 min. at 4° C. Supernatant was transferred to a clean bottle and centrifuged a second time at 4,000 g for 20 min. at 4° C. The resulting lysate was transferred to a new bottle and 0.7 volumes of isopropanol was added and gently mixed for 30 min. Precipitated DNA was pelleted by two rounds of centrifugation at 4,000 g for 30 min. at 4° C., washed with 70% ethanol between the first and second centrifugation. The supernatant was discarded, the DNA pellet was allowed to dry, and the dry DNA was resuspended in 10 mL of TE. The extracted DNA was quantified using an Epoch spectrophotometer, and saved for further processing.

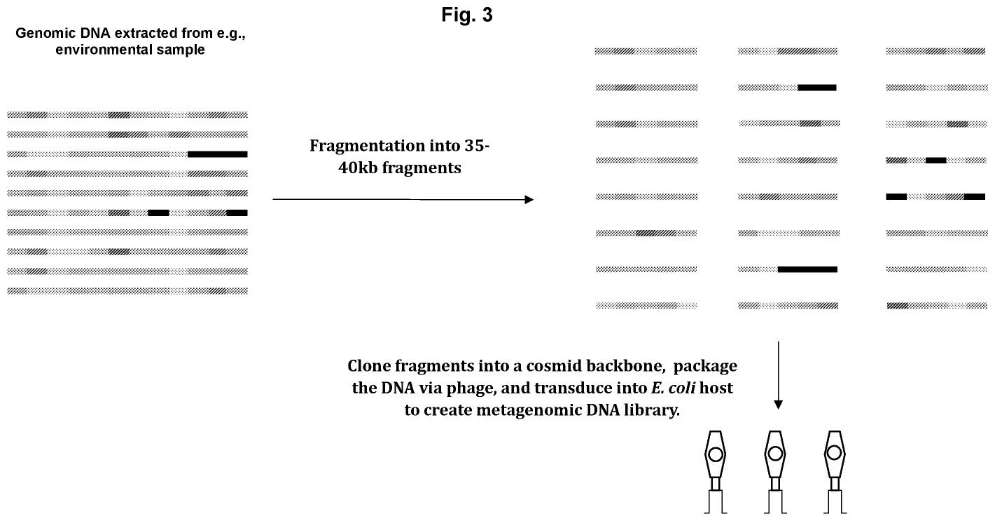

Size selection: Extracted DNA comprising the genomes of the large majority of microorganisms in the soil sample was loaded on an unstained 0.75% agarose gel and separated at constant voltage of 3V/cm for 12-16 hrs. The edges of the gel containing DNA sizing markers were excised and stained. Subsequently, a gel band containing DNA around 35-50 kb was excised. The gel slice containing DNA was placed inside a 12 kD MVVC dialysis tubing with 1× TAE buffer and DNA was electroeluted for 3 hrs at 3V/cm constant voltage. Following electroelution, DNA was concentrated and buffer exchanged into 0.5× TE buffer using a CentriCon ultrafiltration device with 30 kD MVVC membrane. (See FIG. 3).

Cosmid Packaging: DNA was blunt ended using End-It DNA End-Repair kit (Lucigen, ER0720) and isopropanol precipitated. Approximately 10 ligation reactions containing 250 ng of blunt-ended DNA was combined with 500ng of a blunt-ended cosmid vector (T4 ligase, NEB, M0202) and cloned into a cosmid backbone. The cloned DNA was packaged into phages and transduced into E. coli using a MaxPlax™ packaging kit (Lucigen, MP5120) following the manufacturer's instructions. (See FIG. 3). Briefly, packaging extract solution comprising phages were mixed with fragmented DNA by pipetting several times without introducing air bubbles. Reactions were incubated for 90 minutes at 30 Celsius. An additional 25 ul of thawed packaging extract solution was added, and the reactions were incubated for an additional 90 minutes at 30 Celsius. The incubated samples were diluted with Phage Dilution Buffer and gently vortexed. Unincorporated phage proteins were precipitated by adding chloroform a mixing the sample gently. Dilutions were mixed with host E. coli cells, incubated at room temperature for 20 min for phage attachment. The transfected cells were recovered at 37 Celsius for 75 min and plated on LB agar containing appropriate antibiotic selection. Efficiency of packaging was measured for a portion of the packaging reactions according to the manufacturer's instructions.

Pooling and Sequencing: Based on the measured efficiency of packaging, E. coli containing transduced cosmids were combined into pools of roughly 6,000-10,000 cosmids each (“E. coli cosmid pool”). Each E. coli cosmid pool was prepped for sequencing using Nextera XT® DNA Library Prep kit, and sequenced on a HiSeq 4000 or NovaSeq 6000 Illumina® sequencer. (See FIG. 4)

Sequential Assemblies: Reads from pooled samples were trimmed, quality filtered, and paired end reads were merged using BBTools. Contaminating sequences (e.g. cloning vector, host genome) were also removed using BBTools. Clean, merged and unmerged paired end reads were assembled using SPAdes version 3.10.1. The resulting contig assemblies, of average length ˜18 kb were used to prepare longer assemblies across different contigs and pools. (See FIG. 5). The resulting cross-pool assemblies produced large sequence strings, with an average length of ˜32 kb. The assembled sequences were populated into a database and were referred to as a “digital metagenomics library”.

Arraying Physical Pools: E. coli cosmid pools were stored in glycerol in individual cryovials for long term storage. Duplicate E. coli cosmid pools were stored in 96-well format as both glycerol stock of E. coli cells or as isolated DNA from said stock. (See FIG. 6). These were referred to as the “metagenomic physical library.” Each sequence in the digital metagenomics library was associated, via a database, to the location of the corresponding physical DNA fragment within the stored metagenomic physical library.

### Example 2

### Identification of Insecticidal in Metagenomic Libraries (Prophetic Example)

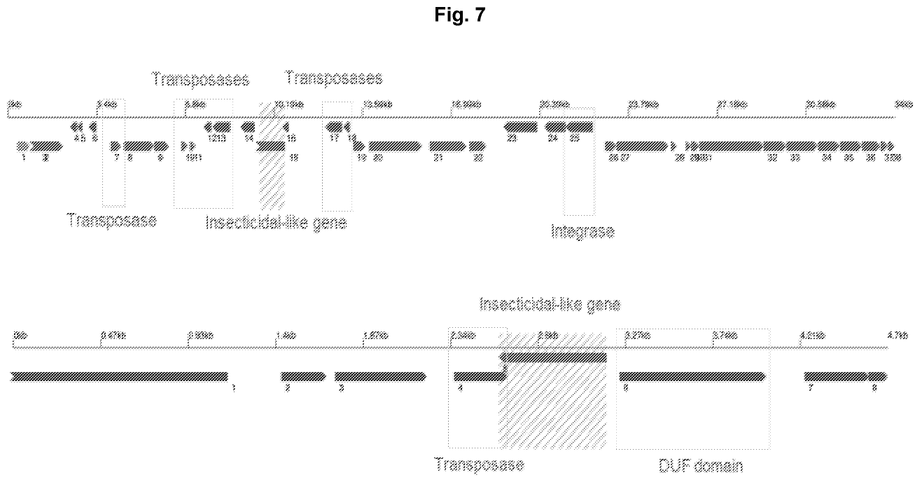

This example illustrates signal based searches of GI-associated digital feature sets for the identification of candidate insecticidals. Insecticidal proteins cluster near signals indicative of GIs (FIG. 7). This example identifies a candidate insecticidal protein using a digital metagenomics library.

HMM libraries (PFAM and TIGRFAM) are searched to identify an appropriate HMM model for a signal indicative of a GI, for example, a gene encoding an integrase, a gene encoding a transposases, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage gene, or a phage gene. If an appropriate model is identified, it will be used. If no model is available, a new HMM model is generated based on a training data set of existing genes (e.g., integrases).

The identified or generated HMM model is used to search for microbial genes encoding a signal of a GI in the digital metagenomics library produced by example 1. The search identifies a series of sequences termed “candidate sequences.” Each candidate sequence is associated with a confidence score assigned by the model. A maximum E value of 1e-10 is established to select top hits for further analysis. In some instances, sequences are de-replicated at 97% identity. Candidate sequences meeting a minimum confidence score are considered signals indicative of a GI, and are advanced to subsequent steps.

All ORFs within a predetermined threshold of 10 kb upstream from the beginning of the signal indicative of a GI and 10 kb downstream of a signal indicative of a GI are compiled into GI-associated digital feature sets. The GI-associated digital feature sets are screened for additional indications of an insecticidal protein encoding gene by assessing for the presence of a pathogenicity factor, such as predicted molecular weight, hydrophobicity, the presence or lack of a transmembrane domain, the presence or lack of other domains of interest, sequence similarity to known insecticidal proteins, sequence similarity to other proteins found in GIs, and predicted taxonomy. ORFs that exhibit an additional indication of an insecticidal encoding gene are advanced to the next step.

DNA comprising ORFs identified above as being located within i) a predetermined threshold of a signal indicative of a GI, and optionally ii) exhibiting an additional indication of an insecticidal encoding gene are recovered from the metagenomics physical library. Briefly, the location of the desired DNA sequence that comprises the ORF is obtained from the metagenomic database, which indicates the plate(s) and well(s) where each sequence from the digital metagenomics library is physically located (i.e., location within the metagenomic physical library). The identified DNA sequences are then recovered from the physical library (e.g., via dilution series to isolate the sequences of interest from the pool), and the DNA sequences that comprise the ORFs are cloned and reassembled in a plasmid vector for expression in a microbial cell, such as E. coli.

The modified microbial host cells are then cultured, and tested for the production of insecticidals, wherein said insecticidals either i) inhibit the growth or development of an insect or ii) kills the insect. Candidate insecticidal proteins are identified by exposing the culture supernatant or expressed protein to an insect. An insecticidal either i) inhibits the growth or development of an insect or ii) kills the insect.

### NUMBERED EMBODIMENTS OF THE DISCLOSURE

Notwithstanding the appended claims, the disclosure sets forth the following numbered embodiments:
1. An in silico method for identifying a candidate insecticidal protein from a digital metagenomics library, said method comprising the steps of:
   a) querying a digital metagenomics library from a microbial population for a signal indicative of a genomic island (GI);
   b) supplying the output of said query as a plurality of GI-associated digital feature sets comprising all gene open reading frames within a predetermined threshold parameter from the signal indicative of the GI;
   c) screening the GI-associated digital feature sets for additional indications of an insecticidal protein encoding gene; and
   d) selecting one or more insecticidal protein encoding gene(s) from among the GI-associated digital feature sets screened in step (c), thereby identifying a candidate insecticidal protein from the digital metagenomics library.
2. The method of embodiment 1, wherein the additional indications of an insecticidal protein encoding gene comprises the presence of a pathogenicity factor within the open reading frame of the GI-associated digital feature sets, said pathogenicity factor selected from the group consisting of: predicted molecular weight, predicted hydrophobicity, presence or lack of transmembrane domain, presence or lack of other domains of interest (e.g. DUF domain), sequence similarity to known insecticidal proteins, sequence similarity to other proteins found in GIs, and predicted taxonomy.
3. The method of embodiment 1, wherein the step of screening of step (c) is conducted by experimentally confirming insecticidal function for a protein encoded by the open reading frame within the GI-associated digital feature sets.
4. The method of embodiment 3, wherein experimental confirmation of insecticidal function is conducted by expressing the protein encoded by the open reading frame within the GI-associated digital features sets in a microbial culture, and exposing said microbial culture to an insect.
5. The method of any one of embodiments 1-4, wherein the predetermined threshold parameter is less 5 kb, 6 kb, 7 kb, 8 kb, 9 kb, or 10 kb from the signal indicative of the GI.
6. The method of any one of embodiments 1-5, wherein the signal indicative of a GI comprises the presence of two or more GI indicators, selected from the group consisting of: a gene encoding an integrase, a gene encoding a transposases, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage and a phage gene.
7. The method of embodiment 6, wherein the GI-associated digital feature sets comprise 3, 4, 5, 6, 7, 8, 9, 10 or more GI indicators.
8. The method of any one of embodiments 1-7, wherein the digital metagenomics library comprises: sequenced and digitally assembled contig sequences having an average length of at least about 10 kb, 15 kb, 20 kb, 25 kb, 30 kb, 35 kb, or 40 kb.
9. The method of any one of embodiments 1-8, wherein the entire digitally metagenomics library is at least about 50 MB, 75 MB, 100 MB, 200 MB, 300 MB, 400 MB, or 500 MB in size.
10. The method of any one of embodiments 1-7, wherein the digital metagenomics library comprises: sequenced and digitally assembled contig sequences having an average length of at least about 10 kb or 20 kb and the entire digitally assembled library is at least about 500 MB in size.
11. The method of any one of embodiments 1-7, wherein the digital metagenomics library comprises: sequenced and digitally assembled contig sequences having an average length of at least about 10 kb or 20 kb and the entire digitally assembled library is at least about 1 TB in size.
12. The method of any one of embodiments 1-7, wherein the digital metagenomics library comprises: sequenced and digitally assembled sequences having an average length of at least about 10 kb or 20 kb and the entire digitally assembled library is about 500 MB to about 1 TB in size.
13. The method of any one of embodiments 6-12, wherein at least one of the GI indicators is identified using an HMM model trained on known sequences comprising the GI indicator.
14. The method of any one of embodiments 2-13, wherein at least one of the pathogenicity factor(s) is identified using an HMM model trained on known sequences comprising the pathogenicity factor.
15. The method of any one of embodiment 1-2 and 5-14, further comprising the step of:
   e) expressing the candidate insecticidal protein in a microbial culture, and exposing the expressed candidate insecticidal protein to an insect.
16. The method of any one of embodiment 1-2 and 5-14, further comprising the step of:
   e) expressing the candidate insecticidal protein in a microbial culture, and exposing said microbial culture to an insect.
17. The method of embodiment 15, wherein the insect is exposed to the microbial culture expressing the candidate insecticidal protein.
18. The method of embodiment 17, wherein the microbial culture is lysed before the insect is exposed to it.
19. The method of any one of embodiments 15-18, wherein the expressed candidate insecticidal protein inhibits the growth or development of the insect.
20. The method of any one of embodiments 15-18, wherein the expressed candidate insecticidal protein kills the insect.
21. The method of any one of embodiments 1-20, wherein the candidate insecticidal protein is part of a multi-gene toxin complex.
22. A method for assembling a deeply sequenced long DNA contig metagenomic library, comprising:
   a) providing an unsequenced and unassembled metagenomic DNA sample comprising whole genomes;
   b) reducing the genomic complexity of the metagenomic DNA sample by:
      i) cloning DNA fragments from the metagenomic library into a plurality of vectors to create a metagenomic vector fragment library that comprises the DNA from the unsequenced and unassembled metagenomic DNA sample;
      ii) pooling the vectors from the metagenomic vector fragment library into a plurality of discrete mini-metagenome subunits that comprise from about 1,000 to about 20,000 pooled vectors each, to create a mini-metagenome library that comprises within the plurality of mini-metagenome subunits the DNA from the unsequenced and unassembled metagenomic DNA sample;
   c) performing intra-pool sequencing and assembly of the metagenomic DNA contained in the pooled vectors present in the plurality of discrete mini-metagenome subunits of the mini-metagenome library to create sequenced and assembled DNA contigs; wherein the average sequenced and assembled DNA contig length is at least about 10 kb, thereby creating a sequenced and assembled intermediary DNA contig length mini-metagenome library; and
   d) optionally performing inter-pool DNA contig assembly, by further assembling a plurality of sequenced and assembled DNA contigs from the intermediary DNA contig length mini-metagenome library to create a long DNA contig length metagenomic library.
23. The method according to embodiment 22, wherein the unsequenced and unassembled metagenomic DNA sample comprises at least about 50, 100, 500, 1000, or 10000, different whole genomes.
24. The method according to embodiment 22, wherein the unweighted average size of the different whole genomes in the unsequenced and unassembled metagenomic DNA sample is at least about 1 MB, 2 MB, 3 MB, 4 MB, or 5 MB.
25. The method according to embodiment 22, wherein the long DNA contig length metagenomic library comprises a plurality of sequenced and assembled DNA contigs with a length of at least about 10 kb, 15 kb, 20 kb, 25 kb, 30 kb, 35 kb, 40 kb, 45 kb, 50 kb, or 100 kb.
26. The method according to embodiment 22, further comprising: arraying the intermediary DNA contig length mini-metagenome library.
27. The method according to embodiment 22, further comprising: arraying the long DNA contig length metagenomic library.
28. The method according to embodiment 22, further comprising: arraying the intermediary DNA contig length mini-metagenome library, or the long DNA contig length metagenomic library, in a bacterial cell or DNA form.
29. The method according to embodiment 22, further comprising: arraying the plurality of discrete mini-metagenome subunits into a real coordinate space and assigning identifiers to each subunit.
30. The method according to embodiment 22, further comprising: arraying the plurality of discrete mini-metagenome subunits into a multi-well microtiter plate.
31. The method according to embodiment 22, further comprising: arraying the plurality of discrete mini-metagenome subunits into a 96-well microtiter plate
32. The method according to embodiment 22, wherein the vectors comprise plasmids.
33. The method according to embodiment 22, wherein the vectors comprise cosmids, fosmids, BACs, YACs, or a combination thereof.
34. The method according to embodiment 22, wherein the vectors comprise cosmids.
35. The method according to embodiment 22, wherein the metagenomic vector fragment library in step b) comprises at least about 1 M or 10 M vectors.
36. The method according to embodiment 22, wherein the vectors comprise cosmids, and the metagenomic vector fragment library in step b) comprises at least about 10 M cosmids.
37. The method according to embodiment 22, wherein the vectors comprise cosmids, and the metagenomic vector fragment library in step b) comprises at least about 20 M cosmids.
38. The method according to embodiment 22, comprising in step b): cloning DNA fragments of less than about 200 kb, from the metagenomic library into a plurality of vectors.
39. The method according to embodiment 22, comprising in step b): cloning DNA fragments of less than about 100 kb, from the metagenomic library into a plurality of vectors.
40. The method according to embodiment 22, comprising in step b): cloning DNA fragments of less than about 50 kb, from the metagenomic library into a plurality of vectors.
41. The method according to embodiment 22, comprising in step b): cloning DNA fragments of about 20 kb to about 50 kb, from the metagenomic library into a plurality of vectors.
42. The method according to embodiment 22, comprising in step b): cloning DNA fragments of about 30 kb to about 45 kb, from the metagenomic library into a plurality of cosmids.
43. The method according to embodiment 22, wherein the discrete mini-metagenome subunits in step b) comprise from about 3,000 to about 15,000 pooled vectors each.
44. The method according to embodiment 22, wherein the discrete mini-metagenome subunits in step b) comprise from about 5,000 to about 12,000 pooled cosmid vectors each.
45. The method according to embodiment 22, wherein the sequenced and assembled intermediary DNA contig length mini-metagenome library comprises contigs with an average length of at least about 10 kb, 15 kb, 20 kb, 25 kb, or 30 kb.
46. The method according to embodiment 22, comprising in step c): simultaneously assembling all the DNA contigs contained in the pooled vectors present in an individual discrete mini-metagenome subunit from the plurality.
47. The method according to embodiment 22, wherein in step c) intra-pool sequencing is performed utilizing single molecule sequencing.
48. The method according to embodiment 22, wherein in step c) intra-pool sequencing is performed utilizing sequencing by synthesis (SBS).
49. The method according to embodiment 22, wherein in step c) intra-pool sequencing is performed utilizing single molecule, real-time (SNRT) sequencing.
50. The method according to embodiment 22, wherein in step c) intra-pool sequencing is performed utilizing nanopore sequencing.
51. The method according to embodiment 22, wherein the discrete mini-metagenome subunits in step b) comprise from about 5,000 to about 12,000 pooled cosmid vectors each, and comprising in step c): simultaneously assembling all the DNA contigs contained in the pooled vectors present in an individual discrete mini-metagenome subunit from the plurality.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following Claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. An in silico method for identifying a candidate insecticidal protein from a digital metagenomics library, said method comprising the steps of:
a) querying a digital metagenomics library from a microbial population for a signal indicative of a genomic island (GI);
b) supplying the output of said query as a plurality of GI-associated digital feature sets comprising all gene open reading frames within a predetermined threshold parameter from the signal indicative of the GI;
c) screening the GI-associated digital feature sets for additional indications of an insecticidal protein encoding gene; and
d) selecting one or more insecticidal protein encoding gene(s) from among the GI-associated digital feature sets screened in step (c), thereby identifying a candidate insecticidal protein from the digital metagenomics library.

2. The method of claim 1, wherein the additional indications of an insecticidal protein encoding gene comprises the presence of a pathogenicity factor within the open reading frame of the GI-associated digital feature sets, said pathogenicity factor selected from the group consisting of: predicted molecular weight, predicted hydrophobicity, presence or lack of transmembrane domain, presence or lack of other domains of interest (e.g. DUF domain), sequence similarity to known insecticidal proteins, sequence similarity to other proteins found in GIs, predicted taxonomy, and insecticidal function.

3. The method of claim 1, wherein the step of screening of step (c) is conducted by experimentally confirming insecticidal function for a protein encoded by the open reading frame within the GI-associated digital feature sets.

4. The method of claim 3, wherein experimental confirmation of insecticidal function is conducted by expressing the protein encoded by the open reading frame within the GI-associated digital features sets in a microbial culture, and exposing said microbial culture to an insect.

5. The method of claim 1, wherein the predetermined threshold parameter is within 10 kb on either end of the signal indicative of the GI.

6. The method of claim 1, wherein the signal indicative of a GI comprises the presence of two or more GI indicators within a single contig, selected from the group consisting of: a gene encoding an integrase, a gene encoding a transposase, a gene encoding an endonuclease, a gene encoding an adhesin, a gene encoding a secretion system, a gene encoding a toxin, a gene encoding an invasion, a gene encoding a modulin, a gene encoding an iron uptake system, a gene encoding a protease, a gene encoding a known insecticidal protein, a gene encoding a virulence factor, a gene encoding a tRNA, a gene containing a domain of unknown function, a prophage and a phage gene.

7. The method of claim 6, wherein the GI-associated digital feature sets comprise 3 or more GI indicators.

8. The method of claim 1, wherein the digital metagenomics library comprises: sequenced and digitally assembled contig sequences having an average or N50 length of at least about 10 kb.

9. The method of claim 1, wherein the digital metagenomics library is at least about 500 MB in size.

10. The method of claim 1, wherein the digital metagenomics library comprises: sequenced and digitally assembled contig sequences having an average or N50 length of at least about 10 kb and the entire digitally assembled library is at least about 500 MB in size.

11. (canceled)

12. (canceled)

13. The method of claim 6, wherein at least one of the GI indicators is identified using an HMM model trained on known GI indicator sequences.

14. The method of claim 2, wherein at least one of the pathogenicity factor(s) is identified using an HMM model trained on known sequences comprising the pathogenicity factor.

15. The method of claim 1, further comprising the step of:
e) expressing the candidate insecticidal protein in a microbial culture, and exposing the expressed candidate insecticidal protein to an insect.

16. The method of claim 1, further comprising the step of:
e) expressing the candidate insecticidal protein in a microbial culture, and exposing said microbial culture to an insect.

17. The method of claim 15, wherein the insect is exposed to the microbial culture expressing the candidate insecticidal protein.

18. The method of claim 17, wherein the microbial culture is lysed before the insect is exposed to it.

19. The method of claim 15, wherein the expressed candidate insecticidal protein inhibits the growth or development of the insect.

20. The method of claim 15, wherein the expressed candidate insecticidal protein kills the insect.

21. The method of claim 1, wherein the candidate insecticidal protein is part of a multi-gene toxin complex.

22. A method for assembling a deeply sequenced long DNA contig metagenomic library, comprising:
a) providing an unsequenced and unassembled metagenomic DNA sample comprising whole genomes;
b) reducing the genomic complexity of the metagenomic DNA sample by:
i) cloning DNA fragments from the metagenomic library into a plurality of vectors to create a metagenomic vector fragment library that comprises the DNA from the unsequenced and unassembled metagenomic DNA sample;
ii) pooling the vectors from the metagenomic vector fragment library into a plurality of discrete mini-metagenome subunits that comprise from about 1,000 to about 20,000 pooled vectors each, to create a mini-metagenome library that comprises within the plurality of mini-metagenome subunits the DNA from the unsequenced and unassembled metagenomic DNA sample;
c) performing intra-pool sequencing and assembly of the metagenomic DNA contained in the pooled vectors present in the plurality of discrete mini-metagenome subunits of the mini-metagenome library to create sequenced and assembled DNA contigs; wherein the average sequenced and assembled DNA contig length is at least about 10 kb, thereby creating a sequenced and assembled intermediary DNA contig length mini-metagenome library; and
d) optionally performing inter-pool DNA contig assembly, by further assembling a plurality of sequenced and assembled DNA contigs from the intermediary DNA contig length mini-metagenome library to create a long DNA contig length metagenomic library.

23-51. (canceled).
